(19) United States
(12) Patent Application Publication
(10) Pub. No.: US 2004/0152745 A1
(43) Pub. Date: Aug. 5, 2004

(54) Dipeptidyl peptidase IV inhibitors and methods of making and using dipeptidyl peptidase IV inhibitors

(75) Inventors: Paul Jackson, Whitehouse Station, NJ (US); Joseph Steiner, Mt. Airy, MD (US)

Correspondence Address: HELLER EHRMAN WHITE & MCAULIFFE, SUITE 300, 1666 K STREET, N.W., WASHINGTON, DC, 20006, US

(73) Assignee: Guilford Pharmaceuticals, Inc., Baltimore, MD

(21) Appl. No.: 10/720,992

(22) Filed: Nov. 25, 2003

Related U.S. Application Data
(63) Continuation of application No. 09/709,383, filed on Nov. 13, 2000, now abandoned.
Application No. 09/709,383 is a continuation-in-part of application No. 09/439,089, filed on Nov. 12, 1999, now abandoned.

(51) Int. Cl. A61K 31/426; A61K 31/421

(52) U.S. Cl. 514/365; 514/374; 514/399

(57) ABSTRACT

The present invention provides the inhibitors of dipeptidyl peptidase IV based upon or including proline or similar moieties. The inhibitors are useful for treating various disorders, including those of the central nervous system and the prostate. Many of the inhibitors can be reversible, and can cross the blood-brain barrier. Methods of making and using the inhibitors and treatment methods also are provided.

This application is a continuation-in-part of U.S. application Ser. No. 09/439,089, filed Nov. 12, 1999, the entirety of which is hereby incorporated by reference.

### BACKGROUND OF THE INVENTION

The present invention relates to new and improved inhibitors of Dipeptidyl Peptidase IV (“DPP IV”), and new and improved treatment methods and related uses. The DPP IV inhibitors according to the invention are useful for treating a wide variety of diseases and other abnormal conditions, including diseases impacting the central nervous system.

Dipeptidyl peptidase IV is a membrane-bound peptidase involved in the release of N-terminal dipeptides from proteins and other types or forms of peptides. The enzyme is a type II membrane serine peptidase, and has a preference for removing proline-containing dipeptides from the N-terminus of the protein or peptide. The enzyme contains 767 amino acids, and has been found in the kidney, epithelial cells, endothelial cells, small intestine, prostrate, seminal plasma and the brain.

The physiological roles of DPP IV have not been completely elucidated. It has been thought that DPP IV plays a role in the cleavage of various cytokines, growth factors and neuropeptides. The enzyme also can cleave neuropeptides such as substance P and neuropeptide Y. There also have been suggestions that DPP IV is involved in cell adhesion and with the T-cell activation marker CD26.

DPP IV has been implicated in disease states such as HIV infection, diabetes, arthritis and certain cancers. For example, a DPP IV presence has been implicated in prostate and lung cancer, and DPP IV also has been found in patients having benign prostate hyperplasia. DPP IV also is being investigated for its role in type II diabetes because the glucagon-like peptide (GLP-1) can be a substrate for DPP IV cleavage, and some DPP IV inhibitors have demonstrated efficacy in animal models for diabetes. Additionally, DPP IV has been implicated in HIV infection due to its association with CD 26. DPP IV also has been identified as a “research front” in an article about Alzheimer's disease. Shvaloff et al., DIALOG FILE NO. 05335738/5.

Inhibition of DPP IV has been shown to increase release of TGF-β, a protein having neuroprotective properties. DPP IV inhibition itself, however, has not been implicated in a neuroprotective context.

DPP IV inhibition has been studied in the treatment of autoimmune diseases such as diabetes, arthritis and multiple sclerosis (a demyelination disease of the peripheral nerves). See PCT publications WO 97/40832 and WO 98/19998. Additionally, PCT publication WO 94/03055 discusses increasing production of hematopoietic cells with DPP IV inhibitors. PCT publication WO 95/11689 discloses the use of DPP IV inhibitors to block the entry of HIV into cells. U.S. Pat. No. 5,543,396 discloses the use of inhibitors (certain proline phosphonate derivatives) to treat tumor invasion. PCT publication WO 95/34538 mentions the use of certain serine protease inhibitors (such as certain DPP IV and PEP inhibitors) to treat peripheral neurological/autoimmune diseases like multiple sclerosis.

DPP IV inhibitors based upon molecules that bear a resemblance to proline have been investigated in the field. For example, PCT publication WO 95/11689 discloses α-amino boronic acid analogs of proline. PCT publication WO 98/19998 discloses N-substituted 2-cyanopyrrolidines as DPP IV inhibitors. PCT publication WO 95/34538 also discloses various proline containing compounds. Alexander et al., BIOSIS NO. 199900218969 discusses research on prolylpyrrolidine phosphonates that are considered irreversible DPP IV inhibitors. U.S. Pat. Nos. 6,011,155; 6,110,949; and 6,124,305 discloses various N-substituted cyanopyrrolidines and cyanothiazolidines to inhibit DPP IV for the treatment of diabetes, and “conditions mediated by dipeptidyl peptidase-V inhibition.”

The field, however, lacks appreciation of the usefulness of DPP IV inhibition for treating disease states, injuries and other abnormal conditions involving the central nervous system and other parts of the body, such as in the treatment of prostate. Therefore, there exists needs for safe and effective compositions and methodologies for treating disease states, injuries and other abnormal conditions involving the central nervous system and other parts of the body by inhibiting DPP IV. These needs have gone unresolved until the development of the present inventions.

### SUMMARY OF THE INVENTIONS

In view of the needs of the art to provide new therapeutic products, methodologies, and uses, it is an object of the invention to provide inhibitors of dipetidyl peptidase.

In accomplishing this object and other objects, there are provided, in accordance with one aspect of the invention, inhibitors of dipeptidyl peptidase IV. The inhibitors according to the invention can include a proline mimetic and preferably possess an IC₅₀ of no more than about 1 μm, preferably no more than 100 nm, and have molecular weights of no more than 700, preferably no more than about 500. Preferably, the inhibitors are reversible. Where the inhibitors are to be used to treat disorders involving the central nervous system, the inhibitors preferably are sufficiently neutral and non-polar such that they can cross the blood-brain barrier via passive diffusion. In many cases, inhibitors that cannot cross by passive diffusion instead cross by active transport. Of course, administration approaches also can be employed when treating the central nervous system to avoid adverse interference from the blood-brain barrier. Inhibitors for use according to the invention include c-KPG and inhibitors according to Core Structures I, II, III or IV, as shown below.

In accordance with another aspect of the present invention, there are provided reversible inhibitors of dipeptidyl peptidase IV, wherein the inhibitor is preferably reversible and preferably has a core structure of selected from the group consisting of Core Structure I, Core Structure II, Core Structure III and Core Structure IV. A given core structure can have functional and substitution groups, such as X, X₁, A, Z and R, wherein X (if present) is CR2R3, O, S, or NR4; X₁ (if present) is CR2R3, O, S, or NR4 with the optional proviso that X and X₁ cannot both be a heteroatom; A is H, COOH, or isosteres of carboxylic acids, such as one selected from the group consisting of CN, SO₃H, CONOH, PO₃R5R6, SO₂NHR7, tetrazole, amides, esters, and acid anhydrides; Z (if present) is O or S; and the various R groups that are present are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, triflouromethyl, or hydroxy, wherein each of R and R1 can be the same or different; all substitutions contemplated herein are permissive for various provisos, either alone or in any combination, such that if one group is included in a given position another group at the same or different position can be excluded; and

In accordance with still another aspect of the invention, there are provided methods of treating patients having disorders involving the central nervous system with inhibitors of DPP IV. Preferably, the inhibitors for use in such methods preferably should be reversible and preferably be able to cross the blood-brain barrier in amounts sufficient to treat the disorder. The compounds according to the invention can be administered concurrently or sequentially with other compounds. Additionally, different compounds according to the invention (e.g., different compounds of one core structure group or compounds of two or more of the core structure groups) can be administered concurrently or sequentially. Uses of the compounds disclosed herein are provided (1) for treating disorders of the central nervous system and (2) for preparing compositions, formulations and medicaments for treating disorders of the central nervous system.

In accordance with still another aspect of the invention, there are provided methods of treating patients having disorders of the prostate, including prostate abnormalities such as prostate cancer and post-prostatectomy nerve recovery. Preferably, the inhibitors for use in such methods should be reversible and be able to penetrate or act upon the prostate. The compounds according to the invention can be administered concurrently or sequentially with other compounds. Additionally, different compounds according to the invention (e.g., different compounds of one core structure group or compounds of two or more of the core structure groups) can be administered concurrently or sequentially. Uses of the compounds disclosed herein are provided (1) for treating disorders of the prostate and (2) for preparing compositions, formulations and medicaments for treating disorders of the prostate.

These and other aspects of the invention will become apparent to the skilled person in view of the teachings contained herein.

### BRIEF DESCRIPTION OF THE DRAWINGS

FIG. 1 graphically depicts an assay employing organotypic spinal motor neurons and threohydroxyaspartate (“THA”). Exposure of neurons with THA alone resulted in death of 55-60% of the neurons. When the neurons were exposed to THA in combination with 10 μM c-KPG, the c-KPG spared greater than 50% of the neurons that would have otherwise been killed.

### DETAILED DESCRIPTION OF PREFERRED EMBODIMENTS

The present invention provides DPP IV inhibitors that are useful for treating various disorders, including those of the central nervous system, among others. Preferably, the DPP IV inhibitors are pyrrolidine-based compounds, and more preferably constitute or include proline or proline mimetics. The compounds according to the present invention preferably have sufficient stability, potency, selectivity, solubility and availability to be safe and effective in treating diseases, injuries and other abnormal conditions or insults to the central nervous system, the peripheral nerves and the prostate, for example. The word “treat” in its various grammatical forms as used in relation to the present invention refers to preventing, curing, reversing, attenuating, alleviating, minimizing, suppressing, ameliorating or halting the deleterious effects of a disease state, disease progression, injury, wound, ischemia, disease causative agent (e.g., bacteria, protozoans, parasites, fungi, viruses, viroids and/or prions), surgical procedure or other abnormal or detrimental condition (all of which are collectively referred to as “disorders,” as will be appreciated by the person of skill in the art). A “therapeutically effective amount” of an inhibitor according to the invention is an amount that can achieve effective treatment, and such amounts can be determined in accordance with the present teachings.

As explained above, DPP IV exhibits a preference for causing the removal of proline-containing dipeptides from the N-terminus of a protein or a peptide. Accordingly, proline has a structure that likely is recognized by or acted upon by the active site of DPP IV. Proline is unique among the 20 naturally-occurring amino acids in that it contains a cyclic secondary amino group, which as a result causes it to create interruptions in alpha-helical structures in proteins or peptides.

Preferably, the DPP IV inhibitors according to the present invention can constitute or include proline or proline-like moieties, often referred to as “proline mimetics.” A proline mimetic is a structure that sufficiently resembles proline such that its charge, polarity, shape and size are sufficiently duplicative of proline so as to participate in many of the molecular interactions involving proline. A molecule or other compound that includes a proline moiety can itself be considered a proline mimetic. Accordingly, molecules that constitute or include proline or proline mimetics can interact with the natural interaction partners of proline, such as DPP IV. Preferably, a DPP IV inhibitor has the same or greater affinity for DPP IV than does the natural substrate of DPP IV, such as a protein containing a proline residue at its N-terminal end. Preferably, the inhibitor will have an equal or greater affinity to permit it to more effectively compete for the active site of DPP IV. Inhibitors with lower affinities, however, are still within the scope of the invention, and effective competition, and thus inhibition, can be ensured through dosing considerations.

In accordance with certain aspects of the invention, the DPP IV inhibitor is used to treat disorders of the prostate, including, but not limited to, prostate cancer and post-prostatectomy nerve recovery. For example, erectile and voiding disorders are extremely common clinical conditions that result from diseases, injuries and trauma including complications associated with pelvic surgery. It is believed that local nerve injury during major pelvic surgeries account for complications such as erectile dysfunction and urinary incontinence. These complications might be caused by the trauma or the injury of the nerves (e.g. cavernous nerve) innervating the area during the surgery. Appropriate administration of a DPP IV inhibitors prior to, during or after surgery may be effective in blocking the nerve degeneration caused by pelvic surgery.

The inhibitor of the invention can be administered in the manner used with other prostate therapeutics, and can be combined with other products or methodologies for treating the prostate. A therapeutically effective amount of the inhibitor will depend upon its potency and its ability to enter or become available at the site of treatment, in this case the prostate and/or surrounding areas. The considerations for determining proper dose levels are available to the skilled person. See Example 6 below.

In accordance with other aspects of the invention, the DPP IV inhibitor can be used to treat disorders of the central nervous system (CNS) and the peripheral nerves. For example, the DPP IV inhibitors according to the present invention can be used to treat CNS maladies such as strokes, tumors, ischemia, Parkinson's disease, memory loss, hearing loss, vision loss, migraines, brain injury, spinal cord injury, Alzheimer's disease and amyotrophic lateral sclerosis (which has a CNS component). Additionally, the DPP IV inhibitors can be used to treat disorders having a more peripheral nature, including multiple sclerosis and diabetic neuropathy.

When treating the CNS, a biological phenomenon known as the “blood-brain barrier” is encountered. The blood-brain barrier prevents many compounds in the circulation from crossing to the brain. The brain is a complex biological structure that is susceptible to a variety of toxins. Additionally, being that the brain is composed primarily of nerves and related tissues, the brain lacks the natural regenerative capabilities of other organs and tissues. For example, the skin has extensive regeneration and restorative capabilities, and thus can withstand encounters with toxins and other physical insults, which it can be expected to encounter in daily life. The brain itself, on the other hand, is quite susceptible to toxins, and thus it is thought that the blood-brain barrier was an evolutionary development to protect the integrity of the brain. The blood-brain barrier, however, also can prevent the entry of beneficial compounds, such as drugs, that are needed to treat a disease, injury or other abnormal condition. Accordingly, the blood-brain barrier can be a complicating factor in developing therapeutics for the CNS.

Compounds, such as molecules, cross the blood-brain barrier by two basic paths, referred to as “passive diffusion” and “active transport.” Designing compounds to cross the blood-brain barrier via passive diffusion is somewhat easier than designing compounds to cross via active transport. Assays for evaluating the capability of a compound to cross the blood-brain barrier are disclosed in Boer et al., DRUG TRANSPORT ACROSS THE BLOOD-BRAIN BARRIER, (Harwood Academic Publishers).

Guidelines exist for creating compounds that cross the blood-brain barrier via passive diffusion. Typically, a compound that crosses the blood-brain barrier via passive diffusion should have a log P between about 1 and about 4. Related to this concept is the log D, which takes into consideration the charge of the compound. Typically, polar and charged compounds are less amenable to crossing the blood-brain barrier by passive diffusion. Accordingly, a log D greater than about −2 is preferred. The concepts of log P and log D are discussed in Waterbeemed, STRUCTURAL-PROPERTY CORRELATIONS IN DRUG RESEARCH (Academic Press).

To further facilitate passive diffusion, the compound preferably has a molecular weight of about 700 or less, preferably about 500 or less. Thus, a compound that is to cross the blood-brain barrier by passive diffusion should be “sufficiently neutral and non-polar” for its size that it can cross the blood-brain barrier in a therapeutically effective amount

Larger and/or more highly charged and polar compounds also are within the scope of the present inventions. Typically, these compounds do not cross the blood-brain barrier via passive diffusion, but rather cross the barrier via active transport. There are guidelines for developing compound that will cross via active transport. Additionally, administration modalities, delivery vehicles and other formulation considerations can assist compounds according to the invention in crossing the blood-brain barrier. See, for example, U.S. Pat. No. 5,874,449.

Besides efficiency of a compound in crossing the blood-brain barrier, another important consideration is the potency of the compound as an inhibitor. For example, potent inhibitors can have a lower efficiency in crossing the blood-brain barrier, but nevertheless can be effective due to their higher potencies. Conversely, a less potent inhibitor may require greater efficiency in crossing the blood-brain barrier in order to have a beneficial effect. Thus, a therapeutically effective amount for treating a CNS disorder depends upon the potency of the inhibitor and its efficiency in crossing the blood-brain barrier or the administration route and approach employed to circumvent the blood-brain barrier.

In terms of potencies, the DPP IV inhibitors preferably have an IC₅₀ (for inhibition concentration where 50% of DPP IV is inhibited) value of less than about 1 μm, and preferably less than 100 nm. Of course, DPP IV inhibitors can have higher IC₅₀ values as long as their efficiency in crossing the blood-brain barrier is sufficient to treat the disease, injury or other abnormal condition.

It is preferred that the DPP IV inhibitor according to the invention is a reversible inhibitor. That is, the DPP IV inhibitor should be able to interact with the inhibitor without becoming permanently bound thereto in a manner that would denature or inactivate the DPP IV enzyme. The need for reversibility is due to the fact that DPP IV is a naturally-occurring enzyme that has normal physiologic functions. An irreversible inhibitor can effectively eliminate functions of the enzyme, and thus result in cessation of normal physiologic processes. The present invention utilizes the inhibition of DPP IV in certain contexts, such as in treating an ischemic event, for definite periods of time, such as during and after reperfusion in the ischemic area. A reversible inhibitor would permit inhibited DPP IV molecules to resume normal function once the need for inhibition is gone.

### Administration Routes and Formulations

For treating the CNS, the compounds according to the invention can be administered by a variety of systemic and CNS-targeted routes. For example, intra-arterial, intravenous intraventricular, intracavitary and intracranial administration routes can be employed. Exemplary injection modalities can be by way of bolus, periodic injection and/or constant infusion.

Depending upon the circumstance, the following routes can be employed for the compounds according to the invention, including parenteral, oral, nasal, inhalation spray, buccally, topically, transdermal, rectal, vaginal, via implanted reservoir or other routes available to the skilled person. The term parenteral as used herein includes subcutaneous, intravenous, intramuscular, intraperitoneal, intrathecal, intraventricular, intrasternal, intracranial or intraosseous injection and infusion techniques.

To be maximally effective as a therapeutic for central nervous system disorders, the compounds of the present invention preferably penetrate the blood-train barrier when peripherally administered. Compounds which cannot sufficiently penetrate the blood-brain barrier can be effectively administered by an intraventricular route. It also is important to note that during the active phase of certain CNS disorders, blood-brain lineage is known to occur and will permit entry of the compounds of the invention to the central nervous system. Moreover, there are several other techniques that either physically break through the blood-brain barrier or circumvent it to deliver therapeutic agents. Examples of these techniques include intrathecal injections, surgical implants, and osmotic techniques. Invasive techniques often are employed, particularly direct administration to damaged neuronal tissue. One or more of the above can be employed according to the invention.

One embodiment for the administration of the compounds of the invention is by intrathecal injection, i.e., directly into the cerebrospinal fluid by puncturing the membranes surrounding the central nervous system is usually by lumbar puncture. Sustained dosages of agents directly into the cerebrospinal fluid can be attained by the use of infusion pumps that are implanted surgically.

Another embodiment for the administration of the compounds of the invention is by injection directly into the lumbar cerebrospinal fluid (intrathecally) or by injection intravenously.

The compounds according to the invention can be formulated with pharmaceutically-acceptable carriers and diluents, and can be used with methods and uses according to the invention. The formulation will depend upon the disease state being treated and the administration route. See, for example, U.S. Pat. No. 5,874,449, which is incorporated by reference. Pharmaceutically acceptable carriers include aqueous solutions, non-toxic excipients, including salts, preservatives, buffers, such as phosphate buffers, and the like, as described in UNITED STATES PHARMACOPEIA AND NATIONAL FORMULARY (USP 24-NF 19); REMINGTON'S PHARMACEUTICAL SCIENCES; HANDBOOK ON PHARMACEUTICAL EXCIPIENTS (2d edition, Wade and Weller eds. 1994), the each of which are hereby incorporated by reference. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles, such as sodium chloride and Ringer's dextrose. Intravenous vehicles include fluid and nutrient replenishers. Preservatives include antimicrobials, anti-oxidants, chelating agents and inert gases. The pH and exact concentration of the various components of the binding composition are adjusted according to routine skills in the art. See GOODMAN AND GILMAN'S THE PHARMACOLOGICAL BASIS FOR THERAPEUTICS (9th edition), the contents of which are hereby incorporated by reference.

Exemplary approaches include those where the compounds are to be administered in the form of sterile injectable preparations, for example, as sterile injectable aqueous or oleaginous suspensions. These suspensions can be formulated according to techniques known in the art using suitable dispersing or wetting agents and suspending agents. The sterile injectable preparations may also be sterile injectable solutions or suspensions in non-toxic parenterally-acceptable diluents or solvents, for example, as solutions in 1,3-butanediol. Among the acceptable vehicles and solvents that may be employed are water, Ringer's solution and isotonic sodium chloride solution. In addition, sterile fixed oils are conventionally employed as solvents or suspending mediums. For this purpose, any blank fixed oil such as a synthetic mono- or di-glyceride may be employed. Fatty acids such a oleic acid and its glyceride derivatives, including olive oil and castor oil, especially in their polyoxyethylated forms, are useful in the preparation of injectables. These oil solutions or suspensions may also contain long-chain alcohol diluents or dispersants.

Additionally, the compounds may be administered orally in the form of capsules, tablets, aqueous suspensions or solutions; Tablets may contain carriers such as lactose and corn starch, and/or lubricating agents such as magnesium stearate. Capsules may contain diluents including lactose and dried corn starch. Aqueous suspensions may contain emulsifying and suspending agents combined with the active ingredient. The oral dosage forms may further contain sweetening and/or flavoring and/or coloring agents.

The compounds may further be administered rectally in the form of suppositories. These compositions can be prepared by mixing the drug with suitable non-irritating excipients which are solid at room temperature, but liquid at rectal temperature such that they will melt in the rectum to release the drug. Such excipients include cocoa butter, beeswax and polyethylene glycols.

Moreover, the compounds may be administered topically, especially when the conditions addressed for treatment involve areas or organs readily accessible by topical application, including neurological disorders of the eye, the skin or the lower intestinal tract.

For topical application to the eye, or ophthalmic use, the compounds can be formulated as micronized suspensions in isotonic, pH adjusted sterile saline or, preferably, as a solution in isotonic, pH adjusted sterile saline, either with or without a preservative such as benzylalkonium chloride. Alternatively, the compounds may be formulated into ointments, such as petrolatum.

For topical application to the skin, the compounds can be formulated into suitable ointments containing the compounds suspended or dissolved in, for example, mixtures with one or more of the following: mineral oil, liquid petrolatum, white petrolatum, propylene glycol, polyoxyethylene polyoxypropylene compound, emulsifying wax and water. Alternatively, the compounds can be formulated into suitable lotions or creams containing the active compound suspended or dissolved in, for example, a mixture of one or more of the following: mineral oil, sorbitan monostearate, polysorbate 60, cetyl ester wax, cetearyl alcohol, 2-octyldodecanol, benzyl alcohol and water.

### Dosing

The compounds of the present invention may be administered by a single dose, multiple discrete doses or continuous infusion. Because the compounds preferably are small, easily diffusible and relatively stable, they can be well-suited to continuous infusion.

Dose levels on the order of about 0.1 mg to about 10,000 mg of the active ingredient are useful in the treatment of the above conditions, with preferred levels being about 0.1 mg to about 1,000 mg. The specific dose level, and thus the therapeutically-effective amount, for any particular patient will vary depending upon a variety of factors, including the activity of the specific compound employed and its bioavailability at the site of drug action; the age, body weight, general health, sex and diet of the patient; the time of administration; the rate of excretion; drug combination; the severity of the particular disease being treated; and the form of administration. Typically, in vitro dosage-effect results provide useful guidance on the proper doses for patient administration. Studies in animal models also are helpful. The considerations for determining the proper dose levels are available to the skilled person. See Example 5 below.

Certain compounds can administered in lyophilized form. In this case, 1 to 100 mg of a compound of the present invention may be lyophilized in individual vials, together with a carrier and a buffer, such as mannitol and sodium phospshate. The compound may be reconstituted in the vials with bacteriostatic water before administration.

In treating CNS disorders resulting from global ischemia, for example, the compounds of the present invention are preferably administered orally, rectally, parenterally or topically at least 1 to 6 times daily, and may follow an initial bolus dose of higher concentration.

### Administration Regimen and Timing

For the compounds methods and uses of the present invention, any administration regimen regulating the timing and sequence of drug delivery can be used and repeated as necessary to effect treatment. Such regimen may include pretreatment and/or co-administration with additional therapeutic agents.

To maximize protection of nervous tissue from nervous insult, the compounds should be administered to the affected cells as soon as possible. In situations where nervous insult is anticipated, the compounds should be administered before the expected nervous insult. Such situations of increased likelihood of nervous insult include surgery (for example, carotid endarterectomy, cardiac, vascular, aortic, orthopedic); endovascular procedures such as arterial catherization (for example, carotid, vertebral, aortic, cardia, renal, spinal, Adamkiewicz); injections of embolic agents; coils or balloons for hemostasis; interruptions of vascularity for treatment of brain lesions; and predisposing medical conditions such as crescendo transient ischemic attacks, emboli and sequential strokes. Where pretreatment for stroke or ischemia is impossible or impracticable, it is important to get the compounds to the affected cells as soon as possible during or after the event. In the time period between strokes, diagnosis and treatment procedures should be minimize to save the cells from further damage and death.

It is clear that both in animal models of stroke and in humans, the effect of cerebral ischemia are manifest on the cerebral metabolism rapidly, with a time scale measured in minutes or hours. Any form of potential neuroprotective treatment should therefore be given by the most rapidly effective route, which in practice usually means intravenously. The optimal duration and route of administration of treatment will depend on the individual pharmacokinetic properties of the neuroprotective compound, on the adverse-effect profile of the drug, and on the nature of the insult that gave rise to the stroke. Excitotoxic injury following stroke evolves over at least 4 hours in rodents and possibly 48 hours in humans. Dyker et al., Stroke 29: 53542 (1998). Thus, it would be desirable to provide neuroprotection throughout this critical time period. Ideally, any compound for the treatment of stroke should adequately cross the blood-brain barrier and obtain sufficiently therapeutic levels within the brain and cerebral spinal fluid.

For patients with prostate cancer that is neither advanced nor metastatic, the compounds of the present invention may be administered (i) prior to surgery or radiation treatment to reduce the risk of metastasis; (ii) during surgery or in conjunction with radiation treatment; and/or (iii) after surgery or radiation therapy to reduce the risk of recurrence and to inhibit the growth of any residual tumorous cells.

For patients with advanced or metastatic prostate cancer, the compounds of the present invention may be administered as a continuous supplement to, or as a replacement for, homonal ablation in order to slow tumor cell growth in both the untreated primary tumor and the existing metastatic lesions.

The compounds, methods and uses of the present invention are particularly useful where shed cells could not be removed by surgical intervention. After post-surgical recovery, the compounds, methods and uses of the present invention would be effective in reducing the chances of recurrence of a tumor engendered by such shed cells.

### Combination with Other Treatments

The compounds, methods and uses of the present invention also to provide combined preparation for simultaneous, separate, or sequential use which contain other biologically active agents.

Such biologically active agent can be either another compound of the present invention; steroids, for example hydrocortisomers such as methylprednisolone; anti-inflammatory or anti-immune drugs, such as methotrexate, azathioprine, cyclophosphamide or cyclosporin A; interferon-β; antibodies, such as anti-CD4 antibodies; agents which can reduce the risk of a second ischemic event, such as ticlopidine; chemotherapeutic compositions; immunotherapeutic compositions; morphine for treating pain; or mixtures thereof.

The compounds according to the invention include various substitutions available to the skilled person and are to be employed in accordance with the teachings contained herein. For example, the Core Structures, which constitute or include proline mimetics, can include a variety of functional groups as taught herein. Additionally, the inventions include isosteres of the compounds or the function groups contained therein. Guiding principles and illustrative examples of functional groups and isosteres are set forth in Smith et al., INTRODUCTION TO THE PRINCIPLES OF DRUG DESIGN (John Wright & Sons, Ltd.), which is hereby incorporated by reference.

The compounds used according to the invention preferably are or contain moieties that resemble proline within their core structures. That is, these compounds are or contain proline mimetics. One such compound that can be used according to the invention contains a proline mimetic and has the following structure:

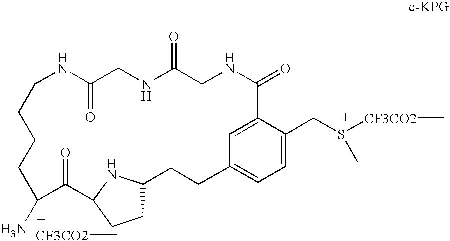

This compound, referred to as “c-KPG,” was tested in an assay employing organotypic spinal motor neurons and threohydroxyaspartate (“THA”), which is an inhibitor of the glutamate reuptake receptor. Synthesis protocols for c-KPG are disclosed in Nguyen et al., J. Med. Chem. 41: 2100-10 (1998).

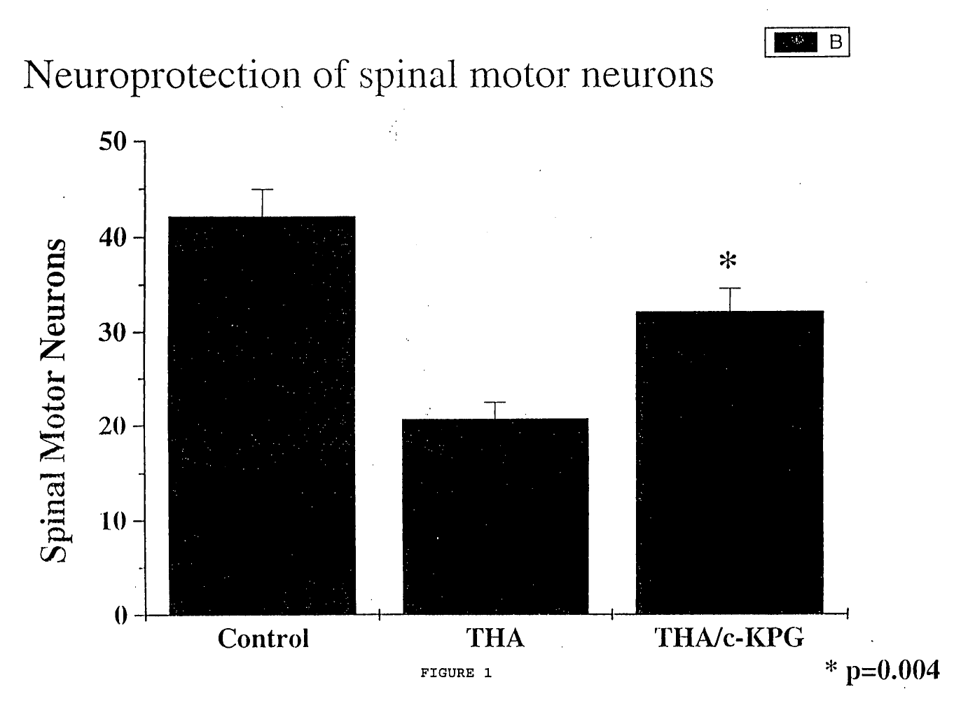

As shown in FIG. 1, exposure of neurons with THA alone resulted in death of 55-60% of the neurons. Exposure of the neurons to THA in combination with 10 μM c-KPG (A DPP IV inhibitor), the c-KPG spared greater than 50% of the neurons that would have otherwise been killed. The results were highly significant (p=0.004).

The invention includes other core structures as well. Core structures, which are DPP IV inhibitors and constitute or contain proline mimetics, are set forth below. Exemplary core structures are depicted schematically, and the functional/substitution groups are set forth in text. All substitutions contemplated herein are permissive for various provisos, either alone or in any combination, such that if one group is included in a given position another group at the same or different position can be excluded. For example, Core Structure I is:

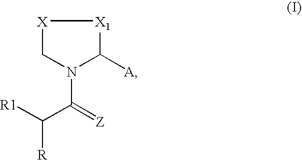

which can be modified as set forth below, with the following numerically-identified optional provisos, which can be employed alone or in any combination:

X is CR2R3, O, S, or NR4; optional proviso 1 that if X is S, then A cannot be CN; optional proviso 2 that if X is CH₂ and R is H, then A cannot be C; optional proviso 3 that if X is S, then R1 cannot be amino-substituted alkyl; optional proviso 4 that if X is CH₂, then A cannot be COOH; optional proviso 5 that if X is S, or if X and X1 are both CH₂, and Z is O, and A is CN, and R1 is H, then R is not NH substituted with C₁-C₉ straight or branched chain alkyl, or NH substituted with C₃-C₇ cycloalkyl;

X₁ is CR2R3, O, S, or NR4 with optional proviso 6 that X and X1 cannot both be a heteroatom; optional proviso 7 if X and X1 are both CH₂, and Z is O, and R1 is NH₂, then R is not 1-methylpropyl if A is COOH, and R is not cyclopentyl if A is CN.

A is H, COOH, or isosteres of carboxylic acids, such as one selected from the group consisting of CN, SO₃H, CONOH, PO₃R5R6, SO₂NHR7, tetrazole, amides, esters, and acid anhydrides with optional proviso 8 that if A is CN, and R1 is NH₂, and Z is O, and R is 1-methylpropyl, then X and X1 are not both CH₂, X and X1 are not S, X is not O, and Z is O or S;

R and R1 are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R and R1 can be the same or different; and

R2, R3, R4, R5, R6 and R7, if present, are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R2, R3, R4, R5, R6 and R7, if present, can be the same or different.

Other core structures are provide according to the invention, such as those having ring modifications (II and III):

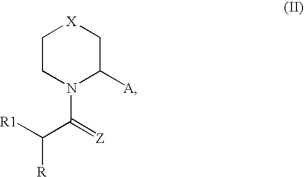

which can be modified as follows:

X is CR2R3, o, S, or NR4;

A is H, COOH, or isosteres of carboxylic acids, such as one selected from the group consisting of CN, SO₃H, CONOH, PO₃R5R6, SO₂NHR7, tetrazole, amides, esters, and acid anhydrides;

Z is O or S;

R and R1 are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R and R1 can be the same or different; and

R2, R3, R4, R5, R6 and R7, if present, are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R2, R3, R4, R5, R6 and R7, if present, can be the same or different.

Core Structure III is:

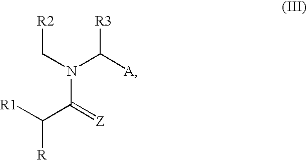

which can be modified as follows:

A is H, COOH, or isosteres of carboxylic acids, such as one selected from the group consisting of CN, SO₃H, CONOH, PO₃R5R6, SO₂NHR7, tetrazole, amides, esters, and acid anhydrides;

Z is O or S;

R, R1, R2 and R3 are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R, R1, R2 and R3 can be the same or different; and

R4, R5, R6 and R7, if present, are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R4, R5, R6 and R7, if present, can be the same or different.

Other compounds according to the invention include amid bond isosteres, such as Core Structure IV. Core Structure IV is:

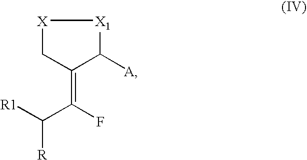

which can be modified as follows:

X is CR2R3, O, S, or NR4;

X, is CR2R3, O, S, or NR4 with the proviso that X and X1 cannot both be a heteroatom;

A is H, COOH, or isosteres of carboxylic acids, such as one selected from the group consisting of CN, SO₃H, CONOH, PO₃R5R6, SO₂NHR7, tetrazole, amides, esters, and acid anhydrides;

R and R1 are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R and R1 can be the same or different; and

R2, R3, R4, R5, R6 and R7, if present, are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R2, R3, R4, R5, R6 and R7, if present, can be the same or different.

The compounds of the core structures according to the present invention can administered in ester or salt forms according to the teachings provided herein. Acceptable formulations, dosages and administration regimens can be determined in accordance with the teachings contained herein.

The invention is further described by the following examples, which are illustrative of the invention but do not limit the invention in any manner.

### EXAMPLE 1

### Synthesis of Compounds According to Core Structure I

Compounds according to Core Structure I can be produced according to a variety of approaches. Representative approaches are shown below:

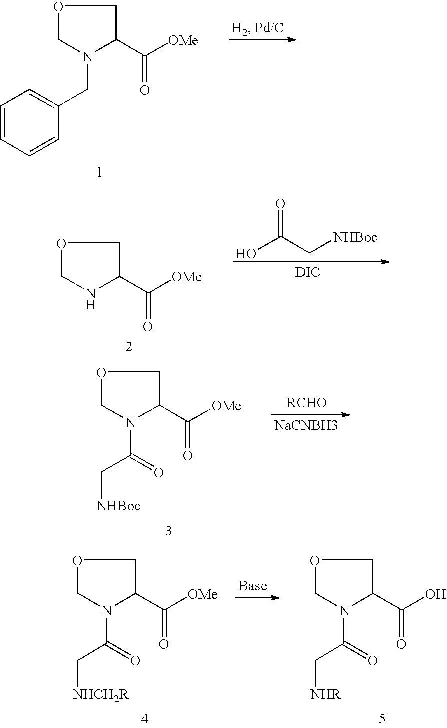

Other approaches include:

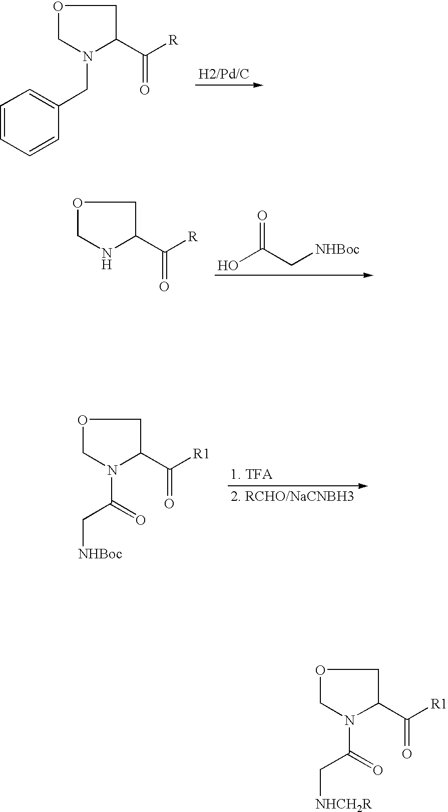

See, for example, Org. Lett. 1: 31-33 (1999).

Substituents can be placed on the ring by modification of starting materials as shown below:

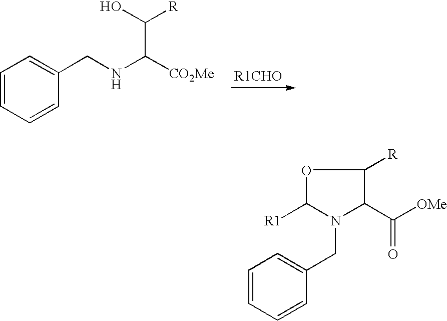

Compounds containing sulfur in place of oxygen can be prepared following standard procedures, as shown below:

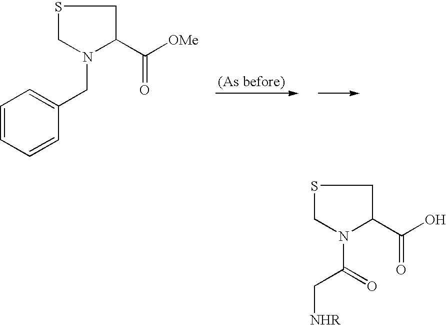

Further transformations can be performed by:

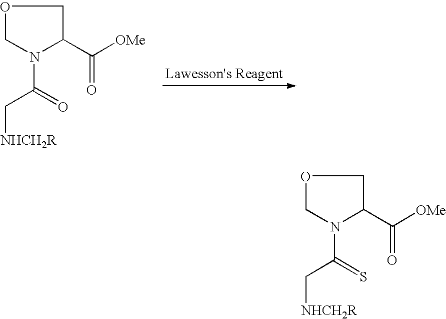

Other exemplary compounds are set forth below.

Compound 1

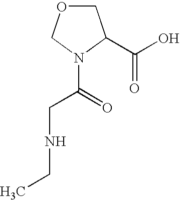

4-oxazolidinecarboxylic acid, 3-[(ethylaminoacetyl]-

| | |
|---|---|
| Principal Group: | Functionalized Hydride: |
| carboxylic acid | 4-oxazolidinecarboxylic acid |
| Parent Hydride: | Substituents: |
| Oxazolidine | 3 acetyl |
| | amino |
| | ethyl |

Compound 2

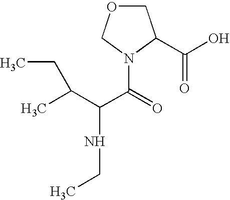

4-oxazolidinecarboxylic acid,
3-[2-(ethylamino)-3-methyl-1 -oxopentyl]-

| | |
|---|---|
| Principal Group: | Functionalized Hydride: |
| carboxylic acid | 4-oxazolidinecarboxylic acid |
| Parent Hydride: | Substituents: |
| Oxazolidine | 3 pentyl |
| | 2 amino |
| | ethyl |
| | 3 methyl |
| | 1 oxo |

Compound 3

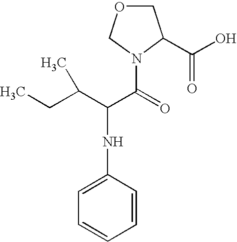

4-oxazolidinecarboxylic acid,
3-[3-methyl-1-oxo-2-(phenylamino)pentyl]-

| | |
|---|---|
| Principal Group: | Functionalized Hydride: |
| carboxylic acid | 4-oxazolidinecarboxylic acid |
| Parent Hydride: | Substituents: |
| Oxazolidine | 3 pentyl |
| | 3 methyl |
| | 1 oxo |
| | 2 amino |
| | phenyl |

Compound 4

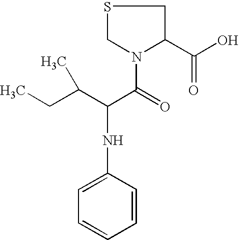

4-thiazolidinecarboxylic acid,
3-[3-methyl-1-oxo-2-(phenylamino)pentyl]-

| | |
|---|---|
| Principal Group: | Functionalized Hydride: |
| carboxylic acid | 4-thiazolidinecarboxylic acid |
| Parent Hydride: | Substituents: |
| thiazolidine | 3 pentyl |
| | 3 methyl |
| | 1 oxo |
| | 2 amino |
| | phenyl |

Compound 5

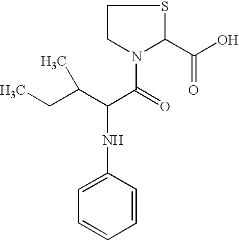

2-thiazolidinecarboxylic acid,
3-[3-methyl-1-oxo-2-(phenylamino)pentyl]-

| | |
|---|---|
| Principal Group: | Functionalized Hydride: |
| carboxylic acid | 2-thiazolidinecarboxylic acid |
| Parent Hydride: | Substituents: |
| thiazolidine | 3 pentyl |
| | 3 methyl |
| | 1 oxo |
| | 2 amino |
| | phenyl |

Compound 6

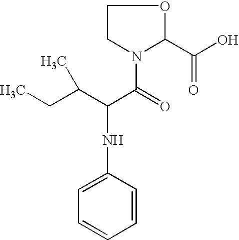

2-oxazolidinecarboxylic acid,
3-[3-methyl-1-oxo-2-(phenylamino)pentyl]-

| | |
|---|---|
| Principal Group: | Functionalized Hydride: |
| carboxylic acid | 2-oxazolidinecarboxylic acid |
| Parent Hydride: | Substituents: |
| Oxazolidine | 3 pentyl |
| | 3 methyl |
| | 1 oxo |
| | 2 amino |
| | phenyl |

Compound 7

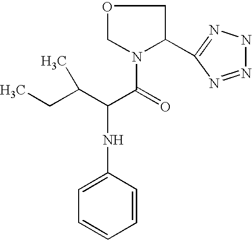

3-oxazolidineethanamine,
α-(1-methylpropyl)-β-oxo-N-phenyl-4-(2H-tetrazol-5-yl)-

| | |
|---|---|
| Principal Group: | Substituents: |
| amine | α propyl |
| Conjunctive Parent: | 1 methyl |
| 3-oxazolidineethanamine | β oxo |
| | N phenyl |
| | 4 2H-tetrazol-5-yl |

Compound 8

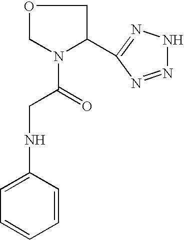

3-oxazolidineethanamine, β-oxo-N-phenyl-4-(2H-tetrazol-5-yl)-

| | |
|---|---|
| Principal Group: | Substituents: |
| amine | β oxo |
| Conjunctive Parent: | N phenyl |
| 3-oxazolidineethanamine | 4 2H-tetrazol-5-yl |

Compound 9

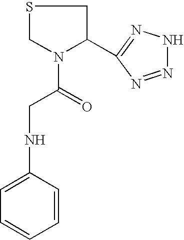

3-thiazolidineethanamine, β-oxo-N-phenyl-4-(2H-tetrazol-5-yl)-

| | |
|---|---|
| Principal Group: | Substituents: |
| amine | β oxo |
| Conjunctive Parent: | N phenyl |
| 3-thiazolidineethanamine | 4 2H-tetrazol-5-yl |

Compound 10

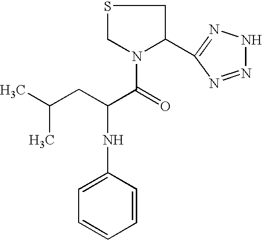

3-thiazolidineethanamine,
α-(2-methylpropyl)-β-oxo-N-phenyl-4-(2H-tetrazol-5-yl)-

| | |
|---|---|
| Principal Group: | Substituents: |
| amine | β oxo |
| Conjunctive Parent: | N phenyl |
| 3-thiazolidineethanamine | 4 2H-tetrazol-5-yl |
| | α propyl |
| | 2 methyl |

Compound 11

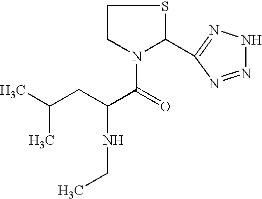

3-thiazolidineethanamine,
N-ethyl-α-(2-methylpropyl)-β-oxo-2-(2H-tetrazol-5-yl)-

| | |
|---|---|
| Principal Group: | Substituents: |
| amine | β oxo |
| Conjunctive Parent: | N ethyl |
| 3-thiazolidineethanamine | 2 2H-tetrazol-5-yl |
| | α propyl |
| | 2 methyl |

Compound 12

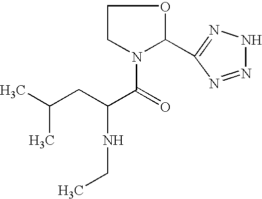

3-oxazolidineethanamine,
N-ethyl-α-(2-methylpropyl)-β-oxo-2-(2H-tetrazol-5-yl)-

| | |
|---|---|
| Principal Group: | Substituents: |
| amine | β oxo |
| Conjunctive Parent: | N ethyl |
| 3-oxazolidineethanamine | 2 2H-tetrazol-5-yl |
| | α propyl |
| | 2 methyl |

Compound 13

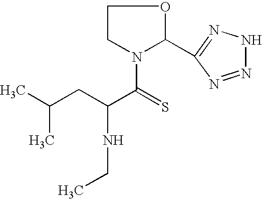

3-oxazolidineethanamine,
N-ethyl-α-(2-methylpropyl)-2-(2H-tetrazol-5-yl)-β-thioxo-

| | |
|---|---|
| Principal Group: | Substituents: |
| amine | β thioxo |
| Conjunctive Parent: | N ethyl |
| 3-oxazolidineethanamine | 2 2H-tetrazol-5-yl |
| | α propyl |
| | 2 methyl |

Compound 14

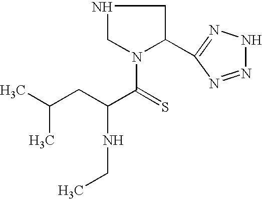

1-imidazolidineethanamine,
N-ethyl-α-(2-methylpropyl)-5-(2H-tetrazol-5-yl)-β-thioxo-

| | |
|---|---|
| Principal Group: | Substituents: |
| amine | β thioxo |
| Conjunctive Parent: | N ethyl |
| 1-imidazolidineethanamine | 5 2H-tetrazol-5-yl |
| | α propyl |
| | 2 methyl |

Compound 15

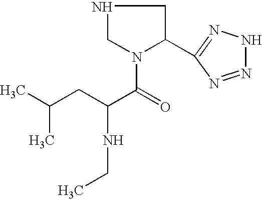

1-imidazolidineethanamine,
N-ethyl-α-(2-methylpropyl)-β-oxo-5-(2H-tetrazol-5-yl) -

| | |
|---|---|
| Principal Group: | Substituents: |
| amine | β oxo |
| Conjunctive Parent: | N ethyl |
| 1-imidazolidineethanamine | 5 2H-tetrazol-5-yl |
| | α propyl |
| | 2 methyl |

Compound 16

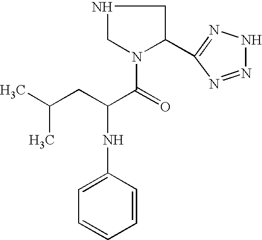

1-imidazolidineethanamine,
α-(2-methylpropyl)-β-oxo-N-phenyl-5-(2H-tetrazol-5-yl) -

| | |
|---|---|
| Principal Group: | Substituents: |
| amine | β oxo |
| Conjunctive Parent: | N phenyl |
| 1-imidazolidineethanamine | 5 2H-tetrazol-5-yl |
| | α propyl |
| | 2 methyl |

Compound 17

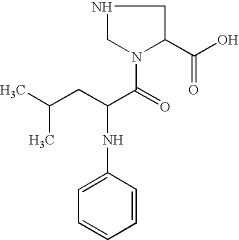

4-imidazolidinecarboxylic acid,
3-[4-methyl-1-oxo-2-(phenylamino)pentyl]-

| | |
|---|---|
| Principal Group: | Substituents: |
| Carboxylic acid | 3 pentyl |
| Parent Hydrid: | 4 methyl |
| Imidazolidine | 1 oxo |
| Functionalized Hydride: | 2 amino |
| 4-imidazolidinecarboxylic acid | phenyl |

Compound 18

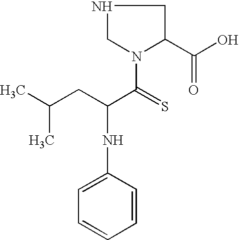

4-imidazolidinecarboxylic acid,
3-[4-methyl-2-(phenylamino)-1-thioxopentyl]-

| | |
|---|---|
| Principal Group: | Substituents: |
| Carboxylic acid | 3 pentyl |
| Parent Hydrid: | 4 methyl |
| Imidazolidine | 2 amino |
| Functionalized Hydride: | 1 thioxo |
| 4-imidazolidinecarboxylic acid | phenyl |

Other compounds for use according to the invention include:

Compound 19: 1-[2-[(5-chloropyridin-2-yl)amino]ethylamino]acetyl-2-cyano-(S)-pyrrolidine dihydrochloride

Compound 20: 1-[2-[(5-trifluoromethylpyridin-2-yl)amino]ethylamino]acetyl-2-cyano-(S)-pyrrolidine

Compound 21 1-[2-[(5-cyanopyridin-2-yl)amino]ethylamino]acetyl-2-cyano-(S)-pyrrolidine dihydrochloride

Compound 22: 1-[2-[(pyrimidin-2-yl)amino]ethylamino]acetyl-2-cyano-(S)-pyrrolidine

Compound 23: 1-[(1-hydroxymethylcyclopent-1-yl)amino]acetyl-2-cyano-(S)-pyrrolidine

Compound 24: 1-[2-[(pyridin-2-yl)amino]ethylamino]acetyl-2-cyano-(S)-pyrrolidine

Compound 25: 1-[2-[(4-chloropyrimidin-2-yl)amino]ethylamino]acetyl-2-cyano-(S)-pyrrolidine

Compound 26: 1-[2-[(3-chloropyridin-2-yl)amino]ethylamino]acetyl-2-cyano-(S)-pyrrolidine

Compound 27: 1-[2-[4-trifluoromethylpyrimidin-2-yl)amino]ethylamino]acetyl-2-cyano-(S)-pyrrolidine

Compound 28: 1-[(2-chlorophenyl)ethylamino]acetyl-2-cyano-(S)-pyrrolidine

Compound 29: 1-[(3,3-diphenyl)propylamino]acetyl-2-cyano-(S)-pyrrolidine

Compound 30: 1-[2-[(5-nitropyridin-2-yl)amino]ethylamino]acetyl-2-cyano-(S)pyrrolidine

Compound 31: 11-[2-[(3-chloro-5-trifluoromethylpyridin-2-yl)amino]ethylamino]acetyl-2-cyano-(S)-pyrrolidine

Compound 32: 11-[2-[(3-trifluoromethylpyridin-2-yl)amino]ethylamino]acetyl-2-cyano-(S) pyrrolidine

Compound 33: 11-[2-[(3,5-dichloropyridin-2-yl)amino]ethylamino]acetyl-2-cyano-(S)-pyrrolidine

Compound 34: 11-[(cyclopent-1-yl)amino-acetyl-2-cyano-(S)-pyrrolidine monohydrochloride

Compound 35: 11-[2-(2-bromo-4,5-dimethoxyphenyl)ethylamino]acetyl-2-cyano-(S)-pyrrolidine

Compound 36: 11-[3-(isopropoxy)propylamino]acetyl-2-cyano-(S)-pyrrolidine monohydrochloride

Compound 37: 11-[(2-hydroxy-1,1-dimethylethylamino)]acetyl-2-cyano-(S)-pyrrolidine monohydrochloride

Compound 38: 11-[3-(2-oxo-pyrrolidin-1-yl)propylamino]acetyl-2-cyano-(S)-pyrrolidine monohydrochloride

Compound 39: 3-[(cyclohexyl)amino]acetyl-4-cyano-R-thiazolidine monohydrochloride

Compound 40: 3-[(3-isopropoxypropyl)amino]acetyl-4-cyano-(R)-thiazolidine monohydrochloride

Compound 41: 3-[(isopropyl)amino]acetyl-4-cyano-(R)-thiazolidine monohydrochloride

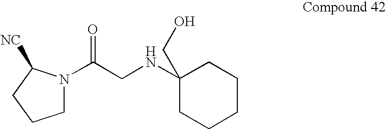

1-[(1-hydroxymethylcyclohexyl)amino]acetyl-2-cyano-(S)-pyrrolidine

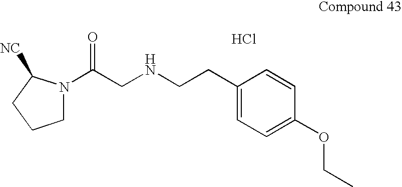

Pyrrolidine, 1-[[2-(4-ethoxyphenyl)ethyl]amino]acetyl-2-cyano-,(S)-,monohydrochloride

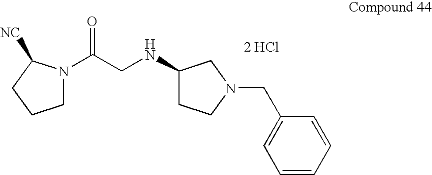

Pyrrolidine, 1-[(1-phenylmethyl-3-prrolidinyl)amino]acetyl-2-cyano-,(S)—(R)-,dihydrochloride

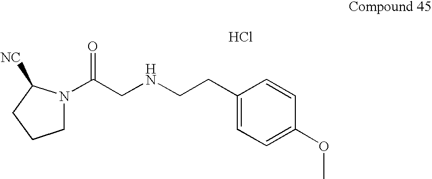

Pyrrolidine, 1-[[2-(4-methoxyphenyl)ethyl]amino]acetyl-2-cyano-,(S)-,monohydrochloride

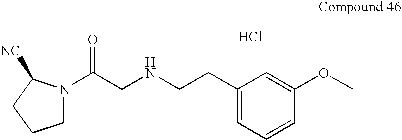

Pyrrolidine, 1-[[2-(3-methoxyphenyl)ethyl]amino]acetyl-2-cyano-,(S)-,monohydrochloride

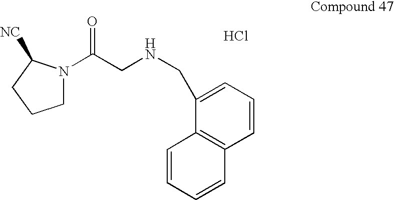

Pyrrolidine, 1-[[(1-naphthalenyl)methyl]amino]acetyl-2-cyano-(S)-,monohydrochloride

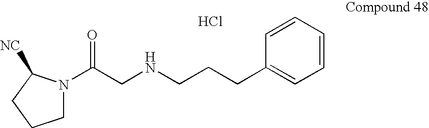

Pyrrolidine, 1-[(3-phenylpropyl)amino]acetyl-2-cyano-,(S)-,monohydrochloride

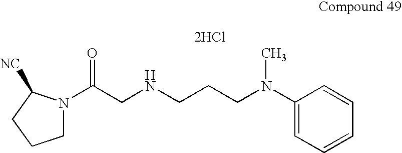

Pyrrolidine, 1-[[3-[(phenyl)(methyl)amino]propyl]amino]acetyl-2-cyano-,(S)-,dihydrochloride

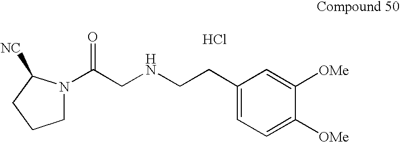

Pyrrolidine, 1-[2-[(3,4-dimethoxyphenyl)ethyl]amino]acetyl-2-cyano-,(S)-,monohydrochloride

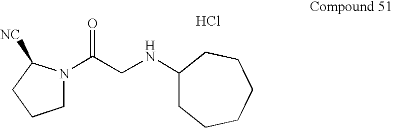

Pyrrolidine, 1-(acycloheptylamino)acetyl-2-cyano-,(S)-,monohydrochloride

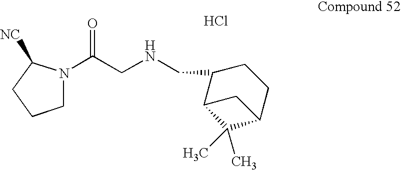

Pyrrolidine, 1-[[(6,6-dimethylbicyclo[3.1.1]hept-2-yl)methyl]amino]acetyl-2-cyano-[1S[1α,2α(S*),5α]]-(S)-,monohydrochloride

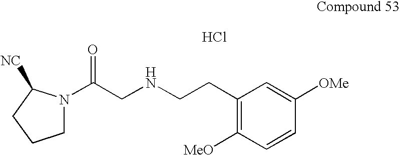

Pyrrolidine, 1-[[2-(2,5-dimethoxyphenyl)ethyl]amino]acetyl-2-cyano-,(S)-,monohydrochloride

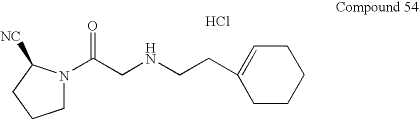

Pyrrolidine, 1-[[2-(1-cyclohexen-1-yl)ethyl]amino]acetyl-2-cyano-,(S)-,monohydrochloride

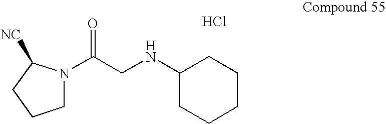

Pyrrolidine, 1-(cyclohexylamino)acetyl-2-cyano-,(S)-,monohydrochloride

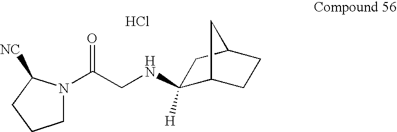

Pyrrolidine, 1-[(bicyclo[2.2.1]hept-2-yl)amino]acetyl-2-cyano-[1S[1α,2α(S*),5α]]-(S)-,monohydrochloride

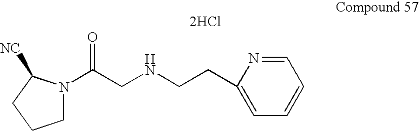

Pyrrolidine, 1-[[2-(2-pyridinyl)ethyl]amino]acetyl-2-cyano-,(S)-,dihydrochloride

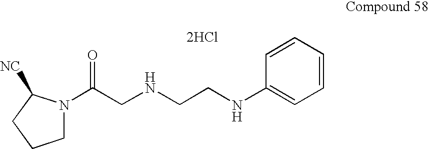

Pyrrolidine, 1-[[(2-phenylamino)ethyl]amino]acetyl-2-cyano-,(S)-,dihydrochloride

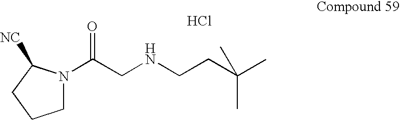

Pyrrolidine, 1-[(3,3-dimethylbutyl)amino]acetyl-2-cyano-,(S)-,monohydrochloride

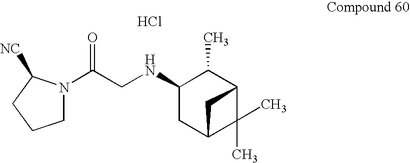

Pyrrolidine, 1-[(2,6,6-trimethylbicyclo[3.1.1]hept-3-yl)amino]acetyl-2-cyano-,(S)[1S[1α,2β,3α(S*),5α]]-monohydrochloride

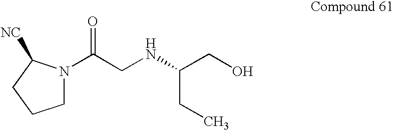

Pyrrolidine, 1[[(1-hydroxymethyl)propyl]amino]acetyl-2-cyano-[S,S)]-

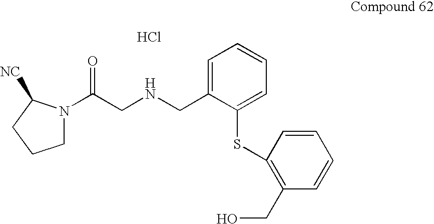

Pyrrolidine, 1-[[[2-[(2-hydroxymethyl)phenyl]thio]phenylmethyl]amino]acetyl-2-cyano-,(S)-,monohydrochloride

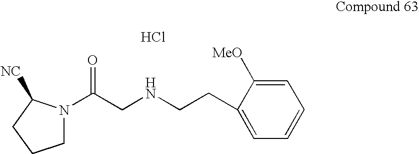

Pyrrolidine, 1-[[2-(2-methoxyphenyl)ethyl]amino]acetyl-2-cyano-,(S)-,monohydrochloride

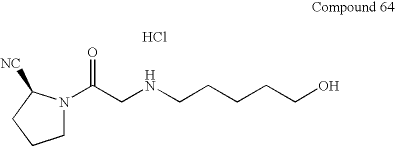

Pyrrolidine, 1-[(5-hydroxypentyl)amino]acetyl-2-cyano-,(S)-,monohydrochloride

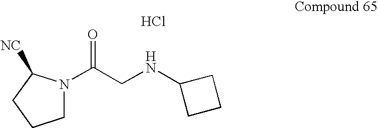

Pyrrolidine, 1-(cyclobutylamino)acetyl-2-cyano-,(S)-monohydrochloride

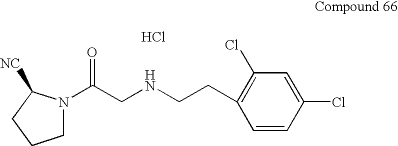

Pyrrolidine, 1-[[2-(2,4-dichlorophenyl)ethyl]amino]acetyl-2-cyano-,(S),monohydrochloride

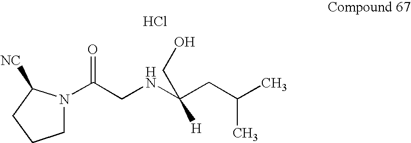

Pyrrolidine, 1-[(1-hydroxymethyl]-3-methylbutyl)amino]acetyl-2-cyano-,O—,(S)—,

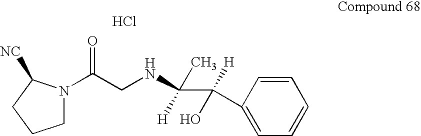

Pyrrolidine, 1-[(2-hydroxy-2-phenylethyl)amino]acetyl-2-cyano-[2S-[1R*,2S*]-monohydrochloride

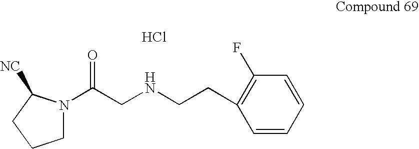

Pyrrolidine, 1-[[2-(2-fluorophenyl)ethyl]amino]acetyl-2-cyano-,(S)-,monohydrochloride

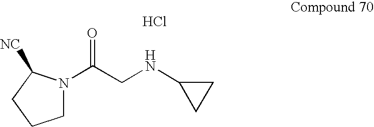

Pyrrolidine, 1-(cyclopropylamino)acetyl-2-cyano-,(S)-,monohydrochloride

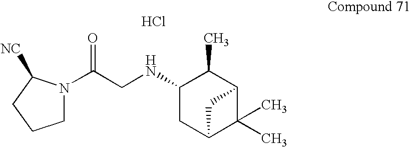

Pyrrolidine, 1-[(2,6,6-trimethylbicyclo[3.1.1]hept-3-yl)amino]acetyl-2-cyano-, [1S[1 alpha, 2 alpha,3 beta (S*),5 alpha]]-monohydrochloride

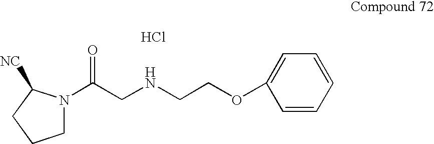

Pyrrolidine, 1-[[(2-phenoxy)ethyl]amino]acetyl-2-cyano-,(S)-,monohydrochloride

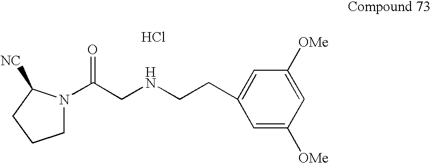

Pyrrolidine, 1-[2-[(3,5-dimethoxyphenyl)ethyl]amino]acetyl-2-cyano-,(S)-,monohydrochloride

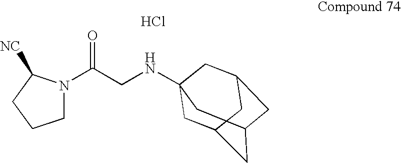

Pyrrolidine, 1-[(1-adamantyl)amino]acetyl-2-cyano-,(S)-,monohydrochloride

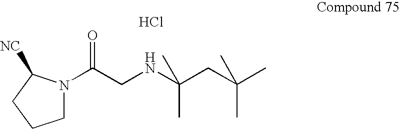

Pyrrolidine, 1-[(1,1,3,3-tetramethylbutyl)amino]acetyl-2-cyano-,(S)-,monohydrochloride

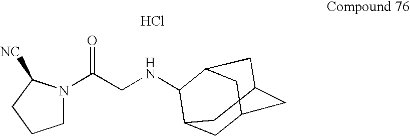

Pyrrolidine, 1-[(2-adamantyl)amino]acetyl-2-cyano-,(S)-,monohydrochloride

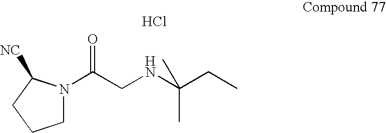

Pyrrolidine, 1-[(1,1-dimethylpropyl)amino]acetyl-2-cyano-,(S)-,monohydrochloride

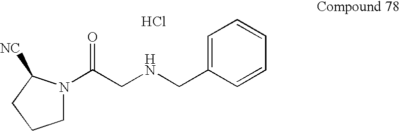

Pyrrolidine, 1-[(phenylmethyl)amino]acetyl-2-cyano-,(S)-,monohydrochloride

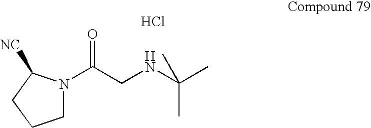

Pyrrolidine, 1-[(1,1-dimethylethyl)amino]acetyl-2-cyano-,(S),monohydrochloride

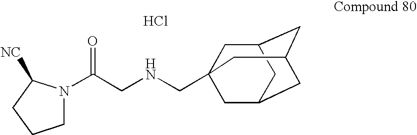

Pyrrolidine, 1-[[(2-adamantyl)methyl]amino]acetyl-2-cyano-,(S)-,monohydrochloride

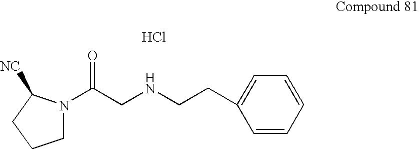

Pyrrolidine, 1-[(2-phenylethyl)amino]acetyl-2-cyano-,(S)-,monohydrochloride

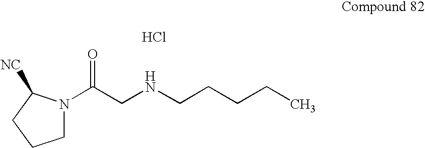

Pyrrolidine, 1-(pentylamino)acetyl-2-cyano-,(S)-,monohydrochloride

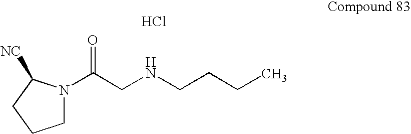

Pyrrolidine, 1-(butylamino)acetyl-2-cyano-,(S)-,monohydrochloride

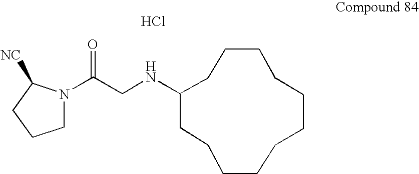

Pyrrolidine, 1-(cyclododecylamino)acetyl-2-cyano-,(S)-,monohydrochloride

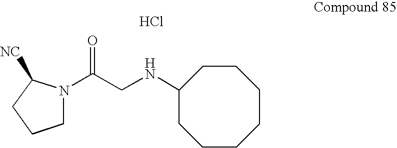

Pyrrolidine, 1-(cyclooctylamino)acetyl-2-cyano-,(S)-,monohydrochloride

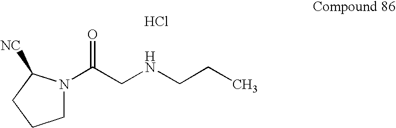

Pyrrolidine, 1-(propylamino)acetyl-2-cyano-,(S)-,monohydrochloride

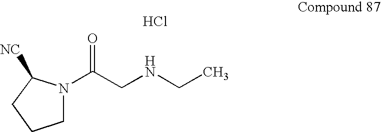

Pyrrolidine, 1-(ethylamino)acetyl-2-cyano-,(S)-,monohydrochloride

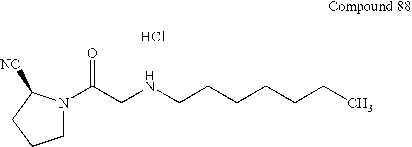

Pyrrolidine, 1-(heptylamino)acetyl-2-cyano-,(S)-,monohydrochloride

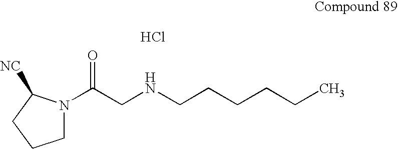

Pyrrolidine, 1-(hexylamino)acetyl-2-cyano-,(S)-,monohydrochloride

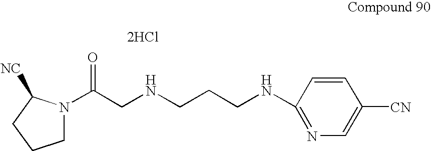

Pyrrolidine, 1-[[3-[(5-cyano-2-pyridinyl)amino]propyl]amino]acetyl-2-cyano-,(S)-,dihydrochloride

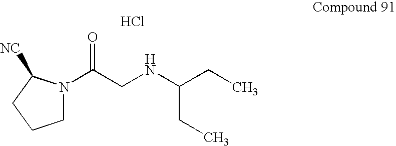

Pyrrolidine, 1-[(1-ethylpropyl)amino]acetyl-2-cyano-,(S)-,monohydrochloride

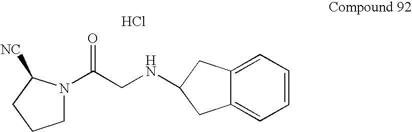

Pyrrolidine, 1-[(2,3-dihydro-1H-inden-2-yl)amino]acetyl-2-cyano-,(S)-,monohydrochloride

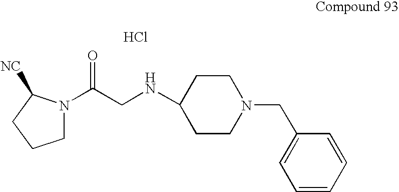

Pyrrolidine, 1-[(1-phenylmethyl-4-piperidinyl)amino]acetyl-2-cyano-,(S)-,-monohydrochloride

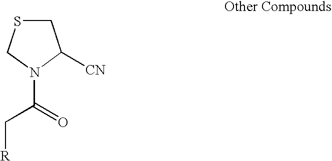

wherein R is NH—R^{I};

R^{I} is: C₁-C₁₂ straight or branched chain alkyl;

C₃-C₇ cycloalkyl;

CH₂—CH₂—NH—R^{II};

CH₂—CH₂—R^{III};

CH₂—CH₂—CHR^{IV}—R^{IV}; or

CH₂—CH₂—CH₂—R^{V};

R^{II} is a pyridine ring optionally substituted in one or two positions with halo, trifluoromethyl, cyano or nitro; or a pyrimidine ring optionally substituted in one position with halo, trifluromethyl, cyano or nitro;

R^{III} is a phenyl ring optionally substituted in one to three positions with halo or C₁-C₃ alkoxy;

Each R^{IV} is independently a phenyl ring optionally substituted in one position with halo or C₁-C₃ alkoxy; and

R^{V} is a 2-oxopyrrolidine group or a C₂-C₄ alkoxy group.

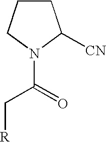

wherein R is NH—R^{I};

R^{I} is: C₁-C₁₂ straight or branched chain alkyl optionally substituted with hydroxy, acetyl,

C₁-C₃ alkoxy, or C₁-C₃ hydroxyalkyl;

C₃-C₁₂ cycloalkyl optionally substituted with hydroxyl, acetyl, C₁-C₃ alkoxy, or

C₁-C₃ hydroxyalkyl;

adamantyl; indanyl; piperidyl optionally substituted with benzyl; pyrrolidine optionally substituted with benzyl; bicycloheptyl optionally substituted in one to three positions with methyl; phenyl optionally substituted with in one to three positions with halo, methoxy, trifluoromethyl; pyridyl optionally substituted in one to three positions with halo, trifluoromethyl, nitro; or pyrimidyl optionally substituted with halo, trifluoromethyl, nitro;

C₁-C₃ straight or branched chain alkyl substituted with R^{IV}, and optionally substituted with hydroxy; or

(CH₂)₁₋₃—NR^{II}R^{III};

R^{II} is hydrogen or methyl;

R^{III} is phenyl optionally substituted with CN, or pyridyl optionally substituted with CN; and

R^{IV} is a group selected from phenyl, naphthyl, cyclohexenyl, pyridyl, pyrimidyl, adamantyl, phenoxy, wherein the group is optionally substituted in one to two positions with ethoxy, methoxy, halo, phenylsulfide, or phenylsulfide substituted with hydroxymethyl.

### EXAMPLE 2

### Synthesis of Compounds According to Core Structure II

Compounds according to Core Structure II can be produced according to a variety of approaches, including the approaches and methodologies provided above for Core Structure I. Appropriate starting materials include:

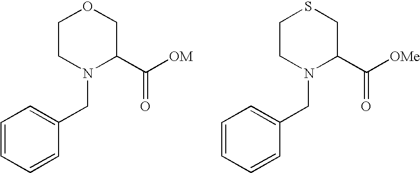

Other synthesis protocols also are available in the art, and are applicable in view of the teachings contained herein. Other exemplary compounds are set forth below.

Compound 1

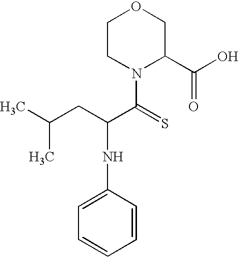

3-morpholinecarboxylic acid,
4-[4-methyl-2-(phenylamino)-1-thioxypentyl]-

| | |
|---|---|
| Principal Group: | Substituents: |
| Carboxylic acid | 4 pentyl |
| Parent Hydrid: | 4 methyl |
| morpholine | 2 amino |
| Functionalized Hydride: | 1 thioxo |
| 3-morpholinecarboxylic acid | phenyl |

Compound 2

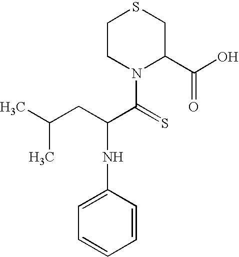

3-thiomorpholinecarboxylic acid,
4-[4-methyl-2-(phenylamino)-1-thioxypentyl]-

| | |
|---|---|
| Principal Group: | Substituents: |
| Carboxylic acid | 4 pentyl |
| Parent Hydrid: | 4 methyl |
| thiomorpholine | 2 amino |
| Functionalized Hydride: | 1 thioxo |
| 3-thiomorpholinecarboxylic acid | phenyl |

Compound 3

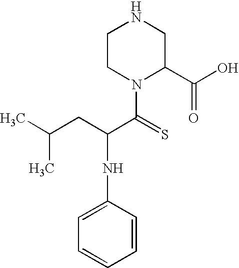

2-piperazinecarboxylic acid,
1-[4-methyl-2-(phenylamino)-1-thioxypentyl]-

| | |
|---|---|
| Principal Group: | Substituents: |
| Carboxylic acid | 1 pentyl |
| Parent Hydrid: | 4 methyl |
| piperazine | 2 amino |
| Functionalized Hydride: | 1 thioxo |
| 2-piperazinecarboxylic acid | phenyl |

Compound 4

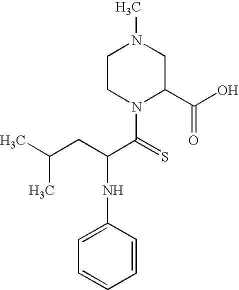

2-piperazinecarboxylic acid,
4-methyl-1-[4-methyl-2-(phenylamino)-1-thioxypentyl]-

| | |
|---|---|
| Principal Group: | Substituents: |
| Carboxylic acid | 1 pentyl |
| Parent Hydnd: | 4 methyl |
| piperazine | 2 amino |
| Functionalized Hydride: | 1 thioxo |
| 2-piperazinecarboxylic acid | phenyl |
| | 4 methyl |

Compound 5

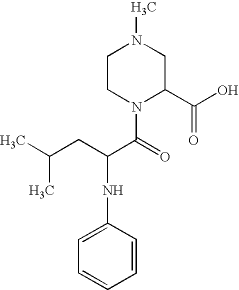

2-piperazinecarboxylic acid,
4-methyl-1-[4-methyl-1-oxo-2-(phenylamino)pentyl]-

| | |
|---|---|
| Principal Group: | Substituents: |
| Carboxylic acid | 1 pentyl |
| Parent Hydrid: | 4 methyl |
| piperazine | 2 amino |
| Functionalized Hydride: | 1 oxo |
| 2-piperazinecarboxylic acid | phenyl |
| | 4 methyl |

Compound 6

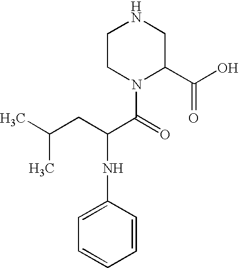

2-piperazinecarboxylic acid,
1-[4-methyl-1-oxo-2-(phenylamino)pentyl]-

| | |
|---|---|
| Principal Group: | Substituents: |
| Carboxylic acid | 1 pentyl |
| Parent Hydrid: | 4 methyl |
| piperazine | 2 amino |
| Functionalized Hydride: | 1 oxo |
| 2-piperazinecarboxylic acid | phenyl |

Compound 7

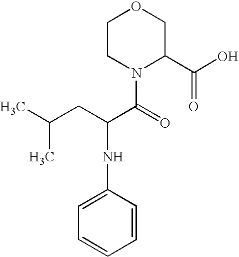

3-morpholinecarboxylic acid,
4-[4-methyl-1-oxo-2-(phenylamino)pentyl]-

| | |
|---|---|
| Principal Group: | Substituents: |
| Carboxylic acid | 4 pentyl |
| Parent Hydrid: | 4 methyl |
| morpholine | 2 amino |
| Functionalized Hydride: | 1 oxo |
| 3-morpholinecarboxylic acid | phenyl |

Compound 8

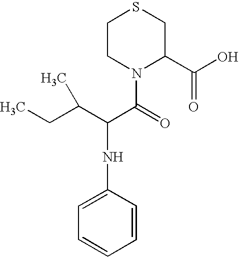

3-thiomorpholinecarboxylic acid,
4-[4-methyl-1-oxo-2-(phenylamino)pentyl]-

| | |
|---|---|
| Principal Group: | Substituents: |
| Carboxylic acid | 4 pentyl |
| Parent Hydrid: | 4 methyl |
| thiomorpholine | 2 amino |
| Functionalized Hydride: | 1 oxo |
| 3-thiomorpholinecarboxylic acid | phenyl |

Compound 9

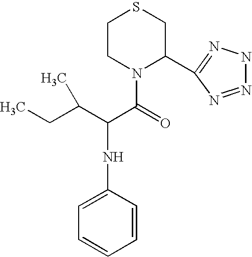

4-thiomorpholineethanamine,
α-(2-methylpropyl)-β-oxo-N-phenyl-3-(2H-tetrazol-5-yl)-

| | |
|---|---|
| Principal Group: | Substituents: |
| amine | α propyl |
| Conjunctive Parent: | 2 methyl |
| 4-thiomorpholineethanamine | 3 2H-tetrazol-5-yl |
| | β oxo |
| | N phenyl |

Compound 10

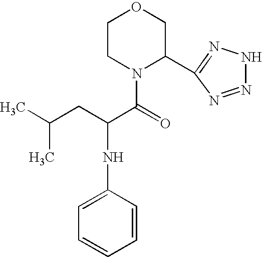

4-morpholineethanamine,
α-(2-methylpropyl)-β-oxo-N-phenyl-3-(2H-tetrazol-5-yl)-

| | |
|---|---|
| Principal Group: | Substituents: |
| amine | α propyl |
| Conjunctive Parent: | 2 methyl |
| 4-morpholineethanamine | 3 2H-tetrazol-5-yl |
| | β oxo |
| | N phenyl |

Compound 11

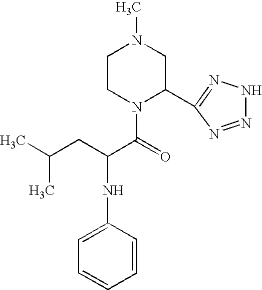

1-piperazineethanamine,
4-methyl-α-(2-methylpronyl)-β-oxo-N-phenyl-2-(2H-tetrazol-5-yl)-

| | |
|---|---|
| Principal Group: | Substituents: |
| amine | α propyl |
| Conjunctive Parent: | 2 methyl |
| 1-piperazineethanamine | 2 2H-tetrazol-5-yl |
| | β oxo |
| | N phenyl |
| | 4 methyl |

Compound 12

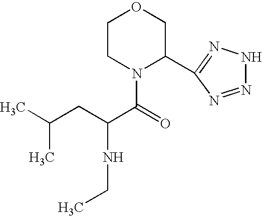

4-morpholineethanamine,
N-ethyl-α-(2-methylpropyl)-β-oxo-3-(2H-tetrazol-5-yl)-

| | |
|---|---|
| Principal Group: | Substituents: |
| amine | α propyl |
| Conjunctive Parent: | 2 methyl |
| 4-morpholineethanamine | 3 2H-tetrazol-5-yI |
| | β oxo |
| | N ethyl |

Compound 13

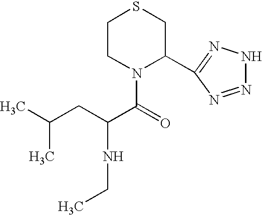

4-thiomorpholineethanamine,
N-ethyl-α-(2-methylpropyl)-β-oxo-3-(2H-tetrazol-5-yl)-

| | |
|---|---|
| Principal Group: | Substituents: |
| amine | α propyl |
| Conjunctive Parent: | 2 methyl |
| 4-thiomorpholineethanamine | 3 2H-tetrazol-5-yl |
| | β oxo |
| | N ethyl |

Compound 14

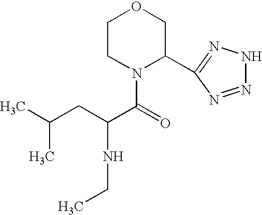

4-morpholineethanamine,
N-ethyl-α-(2-methylpropyl)-β-oxo-3-(2H-tetrazol-5-yl)-

| | |
|---|---|
| Principal Group: | Substituents: |
| amine | α propyl |
| Conjunctive Parent: | 2 methyl |
| 4-morpholineethanamine | 3 2H-tetrazol-5-yl |
| | β oxo |
| | N ethyl |

### EXAMPLE 3

### Synthesis of Compounds According To Core Structure III

Compounds according to Core Structure III can be produced according to a variety of approaches. Representative approaches are shown below:

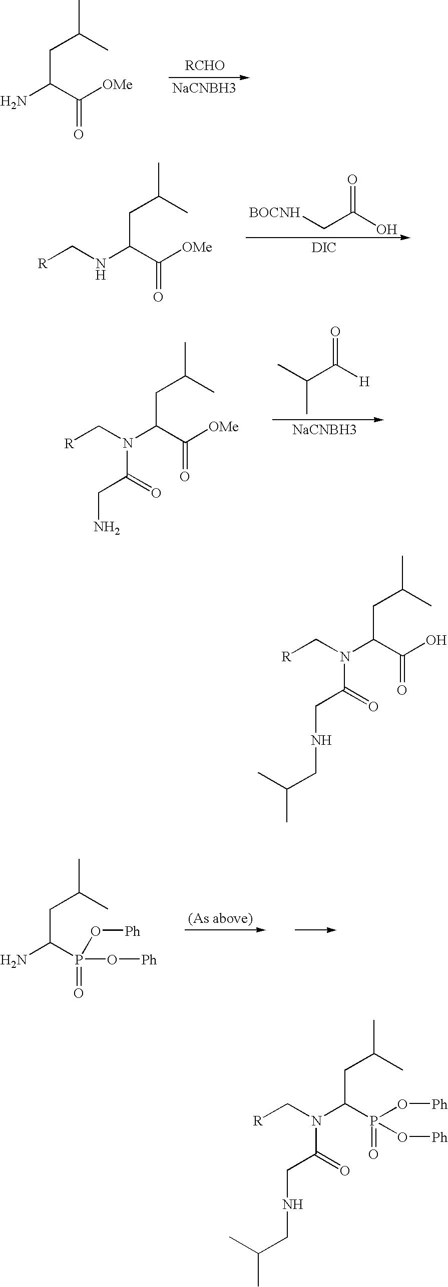

See Oleksyszyn et al., Synthesis 479 (1978).

Other exemplary compounds are depicted below.

Compound 1

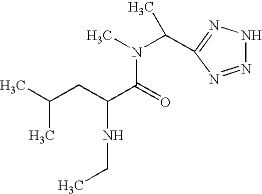

pentamide, 2-(ethylamino)-N,4-dimethyl-N-[1-2H-tetrazol-5-yl)ethyl]-

| | |
|---|---|
| Principal Group: | Substituents: |
| amide | 2 amino |
| Parent Hydrid: | ethyl |
| pentane | N,4-dimethyl |
| Functionalized Hydride: | N ethyl |
| pentamide | 1-2H-tetrazol-5-yl |

Compound 2

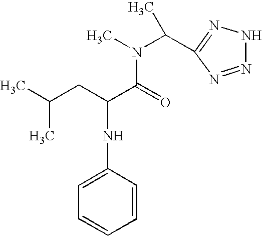

pentamide,
N,4-dimethyl-2-(phenylamino)-N-[1-2H-tetrazol-5-yl)ethyl]-

| | |
|---|---|
| Principal Group: | Substituents: |
| amide | 2 amino |
| Parent Hydrid: | phenyl |
| pentane | N,4-dimethyl |
| Functionalized Hydride: | N ethyl |
| pentamide | 1-2H-tetrazol-5-yl |

Compound 3

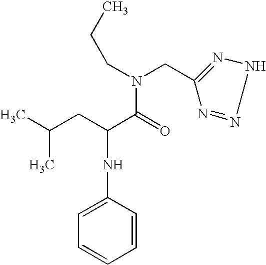

pentamide,
4-methyl-2-(phenylamino)-N-propyl-N-(2H-tetrazol-5-yl methyl)-

| | |
|---|---|
| Principal Group: | Substituents: |
| amide | 4 methyl |
| Parent Hydrid: | 2 amino |
| pentane | phenyl |
| Functionalized Hydride: | N propyl |
| pentamide | N methyl |
| | 2H-tetrazol-5-yl |

Compound 4

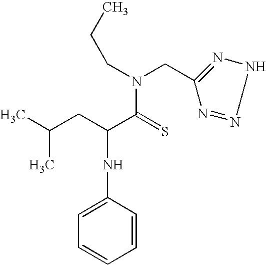

pentanethioamide,
4-methyl-2-(phenylamino)-N-propyl-N-(2H-tetrazol-5-yl methyl)-

| | |
|---|---|
| Principal Group: | Substituents: |
| thioamide | 4 methyl |
| Parent Hydrid: | 2 amino |
| pentane | phenyl |
| Functionalized Hydride: | N propyl |
| pentanethiomide | N methyl |
| | 2H-tetrazol-5-yl |

Compound 5

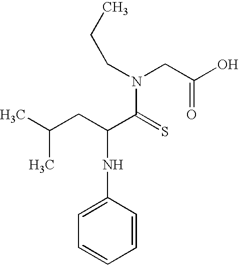

acetic acid,
[[-methyl-2-(phenylamino)-1-thioxopentyl]propylamino]-

| | |
|---|---|
| Principal Group: | Substituents: |
| oic acid | amino |
| Parent Hydrid: | pentyl |
| ethane | 4 methyl |
| Functionalized Hydride: | 2 amino |
| Acetic acid | phenyl |
| | 1 thioxo |
| | propyl |

Compound 6

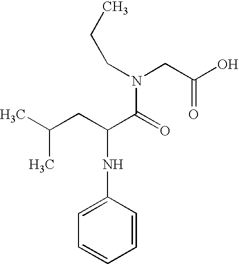

acetic acid,
[[4-methyl-1-oxo-2-(phenylamino)pentyl]propylamino]-

| | |
|---|---|
| Principal Group: | Substituents: |
| oic acid | amino |
| Parent Hydrid: | pentyl |
| ethane | 4 methyl |
| Functionalized Hydride: | 2 amino |
| Acetic acid | phenyl |
| | 1 oxo |
| | propyl |

Compound 7

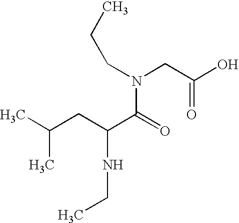

acetic acid, [[2-ethylamino)-4-methyl-1-oxopentyl]propylamino]-

| | |
|---|---|
| Principal Group: | Substituents: |
| oic acid | amino |
| Parent Hydrid: | pentyl |
| ethane | 2 amino |
| Functionalized Hydride: | ethyl |
| Acetic acid | 4 methyl |
| | 1 oxo |
| | propyl |

Compound 8

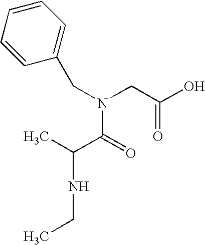

acetic acid, [[2-ethylamino)-1-oxopropyl](phenylmethyl)amino]-

| | |
|---|---|
| Principal Group: | Substituents: |
| oic acid | amino |
| Parent Hydrid: | propyl |
| ethane | 2 amino |
| Functionalized Hydride: | ethyl |
| Acetic acid | 1 oxo |
| | methyl |
| | phenyl |

Compound 9

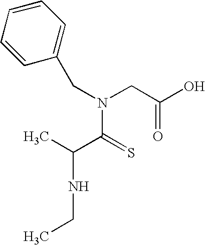

acetic acid, [[2-ethylamino)-1-thioxopropyl](phenylmethyl)amino]-

| | |
|---|---|
| Principal Group: | Substituents: |
| oic acid | amino |
| Parent Hydrid: | propyl |
| ethane | 2 amino |
| Functionalized Hydride: | ethyl |
| Acetic acid | 1 thioxo |
| | methyl |
| | phenyl |

Compound 10

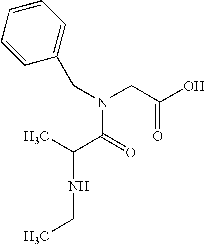

acetic acid, [[2-ethylamino)-1-oxopropyl](phenylmethyl)amino]-

| | |
|---|---|
| Principal Group: | Substituents: |
| oic acid | amino |
| Parent Hydrid: | propyl |
| ethane | 2 amino |
| Functionalized Hydride: | ethyl |
| Acetic acid | 1 oxo |
| | methyl |
| | phenyl |

Compound 11

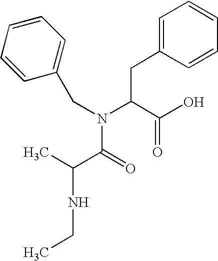

phenylalanine, N-[2-ethylamino)-1-oxopropyl]-N-(phenylmethyl)-

| | |
|---|---|
| Principal Group: | Substituents: |
| oic acid | N-propyl |
| Conjunctive Parent: | 2 amino |
| phenylalanine | ethyl |
| | 1 oxo |
| | N methyl |
| | Phenyl |

Compound 12

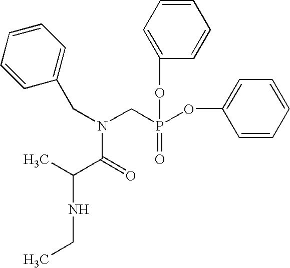

phosphonic acid,
[[[2-(ethylamino)-1-oxopropyl](phenylmethyl)amino]methyl]-,
diphenyl ester-

| | |
|---|---|
| Principal Group: | Substituents: |
| phosphonic acid | methyl |
| Modifiers: | amino |
| diphenyl | propyl |
| | 2 amino |
| | ethyl |
| | 1 oxo |
| | methyl |
| | phenyl |

Compound 13

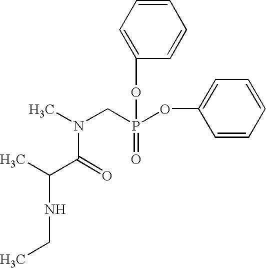

phosphonic acid,
[[[2-(ethylamino)-1-oxopropyl]methylamino]methyl]-,
diphenyl ester

| | |
|---|---|
| Principal Group: | Substituents: |
| phosphonic acid | methyl |
| Modifiers: | amino |
| diphenyl | propyl |
| | 2 amino |
| | ethyl |
| | 1 oxo |
| | methyl |

### EXAMPLE 4

### Synthesis of Compounds According to Core Structure IV

Methodologies for production of compounds according to Core Structure IV are disclosed in Lin et al., Proc. Nat'l Acad. Sci. USA 95: 14020-24 (1998). Exemplary compounds are set forth below.

Compound 1

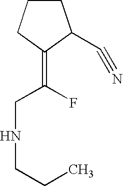

cyclopentanecarbonitrile,
2-[1-fluoro-2-(propylamino)ethylidene]-, (2Z)-

Principal Group:
carbanitrile
Parent Hydride:
cyclopentane
Functionalized Hydride:
cyclopentanecarbonitrile
Substituents:
2 ethylidene
1 fluoro
2 amino
propyl

Compound 2

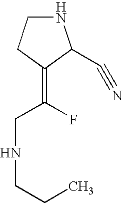

2-pyrrolidinecarbonitrile,
3-[1-fluoro-2-(propylamino)ethylidene]-, (3Z)-

Principal Group:
carbonitrile
Parent Hydride:
pyrrolidine
Functionalized Hydride:
2-pyrrolidinecarbonitrile
Substituents:
3 ethylidene
1 fluoro
2 amino
propyl

Compound 3

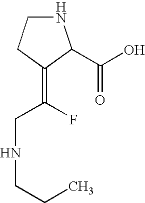

2-pyrrolidinecarboxylic acid,
3-[1-fluoro-2-(propylamino)ethylidene]-, (3Z)-

Principal Group:
carboxylic acid
Parent Hydride:
pyrrolidine
Functionalized Hydride:
2-pyrrolidinecarboxylic acid
Substituents:
3 ethylidane
1 fluoro
2 amino
propyl

Compound 4

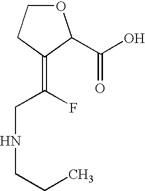

2-furancarboxyli acid,
3-[1-fluoro-2-(propylamino)ethylidene]tetrahydro-, (3Z)-

Principal Group:
carboxylic acid
Parent Hydride:
furan
Functionalized Hydride:
2-furancarboxylic acid
Substituents:
3 ethylidene
1 fluoro
2 amino
propyl
tetrahydro

Compound 5

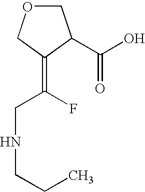

3-furancarboxylic acid,
4-[1-fluoro-2-(propylamino)ethylidene]tetrahydro-, (4E)-

Principal Group:
carboxylic acid
Parent Hydride:
furan
Functionalized Hydride:
3-furancarboxylic acid
Substituents:
4 ethylidene
1 fluoro
2 amino
propyl
tetrahydro

Compound 6

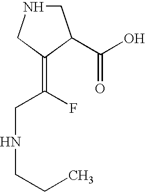

3-pyrrolidinecarboxylic acid,
4-[1-fluoro-2-(propylamino)ethylidene]-, (4E)-

Principal Group:
carboxylic acid
Parent Hydride:
pyrrolidine
Functionalized Hydride:
3-pyrrolidinecarboxylic acid
Substituents:
4 ethylidene
1 fluoro
2 amino
propyl

Compound 7

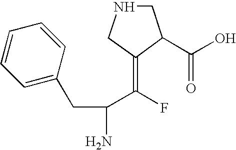

3-pyrrolidinecarboxylic acid,
4-(2-amino-1-fluoro-3-phenylpropylidene)-, (4E)-

Principal Group:
carboxylic acid
Parent Hydride:
pyrrolidine
Functionalized Hydride:
3-pyrrolidinecarboxylic acid
Substituents:
4 propylidene
2 amino
1 fluoro
3 phenyl

Compound 8

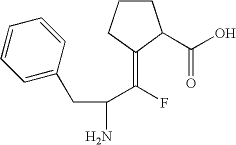

cyclopentanecarboxylic acid,
2-(2-amino-1-fluoro-3-phenylpropylidene)-, (2Z)-

Principal Group:
carboxylic acid
Parent Hydride:
cyctopentane
Functionalized Hydride:
cyclopentanecarboxylic acid
Substituents:
2 propylidene
2 amino
1 fluoro
3 phenyl

Compound 9

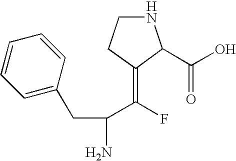

2-pyrrolidinecarboxylic acid,
3-(2-amino-1-fluoro-3-phenylpropylidene)-, (3Z)-

Principal Group:
carboxylic acid
Parent Hydride:
pyrrolidine
Functionalized Hydride:
2-pyrrolidinecarboxylic acid
Substituents:
3 propylidene
2 amino
1 fluoro
3 phenyl

Compound 10

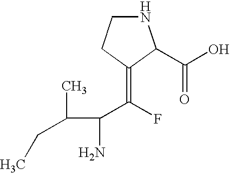

2-pyrrolidinecarboxylic acid,
3-(2-amino-1-fluoro-3-methylpentylidene)-, (3Z)-

Principal Group:
carboxylic acid
Parent Hydride:
pyrrolidine
Functionalized Hydride:
2-pyrrolidinecarboxylic acid
Substituents:
3 pentylidene
2 amino
1 fluoro
3 methyl

Compound 11

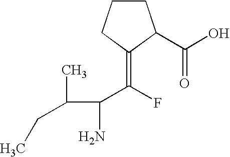

cyclopentanecarboxylic acid,
2-(2-amino-1-fluoro-3-methylpentylidene), (2Z)-

Principal Group:
carboxylic acid
Parent Hydride:
cyclopentane
Functionalized Hydride:
cyclopentanecarboxylic acid
Substituents:
2 pentylidene
2 amino
1 fluoro
3 methyl

Compound 12

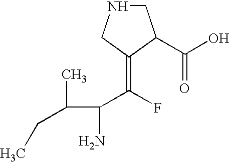

3-pyrrolidinecarboxylic acid,
4-(2-amino-1-fluoro-3-methylpentylidene)-, (4E)-

Principal Group:
carboxylic actd
Parent Hydride:
pyrrolidine
Functionalized Hydride:
3-pyrrolidinecarboxylic acid
Substituents:
4 pentylidene
2 amino
1 fluoro
3 methyl

Compound 13

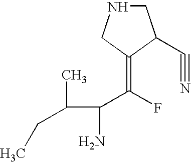

3-pyrrolidinecarbonitrile,
4-(2-amino-1-fluoro-3-methylpentylidene)-, (4E)-

Principal Group:
carbonitrite
Parent Hydride:
pyrrolidine
Functionalized Hydride:
3-pyrrolidinecarbonitrile
Substituents:
4 pentylidene
2 amino
1 fluoro
3 methyl

Compound 14

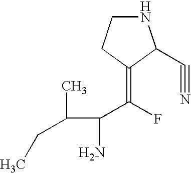

2-pyrrolidinecarbonitrile,
3-(2-amino-1-fluoro-3-methylpentylidene)-, (3Z)-

Principal Group:
carbonitrile
Parent Hydride:
pyrrolidine
Functionalized Hydride:
2-pyrrolidinecarbonitrile
Substituents:
3 pentylidene
2 amino
1 fluoro
3 methyl

Compound 15

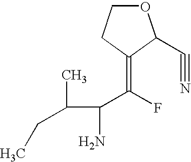

2-furancarbonitrile,
3-(2-amino-1-fluoro-3-methylpentylidene)tetrahydro-, (3Z)-

Principal Group:
carbonitrile
Parent Hydride:
furan
Functionalized Hydride:
2-furancarbonitrile
Substituents:
3 pentylidene
2 amino
1 fluoro
3 methyl
tetrahydro

Compound 16

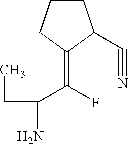

cyclopentanecarbonitrile, 2-(2-amino-1-fluorobutylidene)-, (2Z)-

Principal Group:
carbonitrile
Parent Hydride:
cyclopentane
Functionalized Hydride:
cyclopentanecarbonitrile
Substituents:
2 butylidene
2 amino
1 fluoro

Compound 17

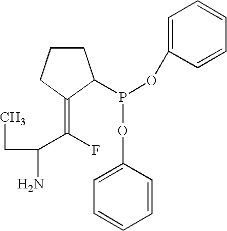

phosphonous acid,
[(2Z)-2-(2-amino-1-fluorobutylidene)cyclopentyl]-, diphenyl ester

Parent Hydride:
phosphonous acid
Substituents:
cyclopentyl
2 butytidene
2 amino
1 fluoro
Modifiers:
diphenyl

Compound 18

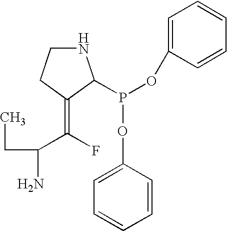

phosphonous acid,
[(3Z)-3-(2-amino-1-fluorobutylidene)pyrrolidinyl]-, diphenyl ester

Parent Hydride;
phosphonous acid
Substituents:
pyrralidinyl
3 butylidene
2 amino
1 fluoro
Modifiers:
diphenyl

Compound 19

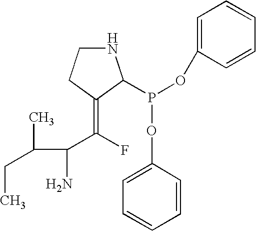

phosphonous acid,
[(3Z)-3-(2-amino-1-fluoro-3-methylpentylidene)pyrrolidinyl]-,
diphenyl ester

Parent Hydride:
phosphonous acid
Substituents:
pyrrolidinyl
3 pentylidene
2 amino
1 fluoro
3 methyl
Modifiers:
diphenyl

Compound 20

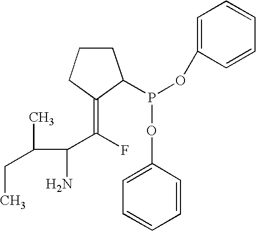

phosphonous acid,
[(2Z)-2-(2-amino-1-fluoro-3-methylpentylidene)cyclopentyl]-,
diphenyl ester

Parent Hydride:
phosphonous acid
Substituents:
cyclopentyl
2 pentylidene
2 amino
1 fluoro
3 methyl
Modifiers:
diphenyl

### EXAMPLE 5

### Exemplary Neuroactivity Testing Protocols

There are a variety of protocols available for evaluating the neuroactivity of the above compounds and other compounds designed, made and used according to the invention. These assays can be in vivo or in vitro methods. The approaches below include assays measuring the ability of compounds to protect neuronal cells from toxic treatments, and the ability of the compounds to elicit neuronal cell growth, regeneration, neurite extension and the like.

Immunostaining and Neurite Outgrowth Quantitation Assays

Spinal cord and dorsal root ganglion (DRG) cells from adult mice can be isolated by micro-dissection. The spinal cord with attached DRGs from an adult mouse (15-10 g) is removed. Spinal nerves are cut away using micro-dissection scissors and any excess material is trimmed until the DRG is free. Using sharp micro-dissecting scissors, a transverse cut is made in the peripheral nerve, leaving 1-2 mm attached, and the explant is placed into Petri dish and covered with plating media. When finished collecting all DRGs, the spinal nerve is trimmed to about 1 mM in length. Then, embed the explant in 30 μL of reduced growth factor Matrigel on a circular coverslip, and place in a 35 mM culture dish. Cover the sensory ganglion explant with 2 mls of media. Compounds, drugs or control solutions are added from 10X stocks, and are incubated at 37° C., 5% CO₂, 95% humidity for 48 hrs. Wash cultures twice with PBS, and fix with 10% formalin for 30 minutes. Wash the fixed cultures twice with PBS and store refrigerated in PBS.

Place cultures in Block Buffer (5% Horse Serum, 5% Goat Serum, 1% Triton X, PBS pH=7.4) overnight, while rotating, at a temperature of 4° C. Add primary antibody (for example, Beta tubulin, Sigma Chemical Co.) diluted in Block Buffer and incubate overnight at 4° C. Wash 5 times with PBS and apply secondary antibody (Alexa 488 Goat Anti-Mouse) diluted in block buffer. Incubate overnight at 4° C. Wash 5 times with PBS and leave overnight at 4° C. Coverslip the cultures and measure total neurite length from the end of the attached spinal nerve. Lengths of all neurites are quantitated and compared to those present in vehicle-treated control DRGs.

Neuroprotection Assays

Cultures are derived from postnatal day 8 (P8) Sprague-Dawley rat lumbar spinal cord slices of 325 micron thickness. Each experiment consists of two 6-well plates with 5 slices from 4 different animals per well. Media changes are performed every 3 to 4 days. Cultures are treated with THA [L(−)-threo-3-hydroxyaspartic acid; Tocris Cookson Inc., Ballwin, Mo.] at 200 μM+compound (10 μM) after one week in culture. The control is an untreated sample with 0.1% DMSO as vehicle. The THA control is a THA treated sample with 0.1% DSMO as vehicle. Two wells are used per condition. One media change with new THA and compounds is performed. The experiment is stopped 6 to 8 days following drug treatment (13-15 total days in vitro, DIV) as dictated by visual assessment of lesion, by fixation with 4% paraformaldehyde/0.1 M phosphate buffer for 30 minutes. Slices are permeabilized with 100% cold methanol for 10 minutes. Slices are transferred to staining wells. The slices are blocked with 10% HS/TBS. Primary antibody incubation is overnight at 4° C. with SMI-32 antibody 1:5000 in 2% HS/TBS. SMI-32 was specific towards unphosphorylated H neurofilament subunit. Vectastain ABC Elite Kit with rat absorbed anti-mouse secondary antibody is used with DAB to stain the slices. The slices are mounted onto a slide and a coverslip is sealed with DPX mounting solution.

Quantification of surviving neurons is performed on a Ziess Axiovert microscope. Neuronal survival is determined by observing an intact neuronal cell body with processes located ventrally of the central canal in each hemisphere. This correlates to laminae VII, VIII and IX. Each hemisphere is counted individually. The statistics can be performed with StatView software on a minimum of three different experiments per condition and significance should be determined as compared to THA control. The percent of protection can be determined from the average number of living neurons by the following equation:

(drug treatment condition—THA control)/(Untreated control—THA control).

### EXAMPLE 6

### Exemplary Testing Protocols For Prostate Treatment Efficacy

Protocols for testing efficacy, dosing, and administration schedules for post-prostatectomy nerve recovery can be performed in accordance with the teachings of Example 5.

To evaluate DPP IV inhibitors in the treatment of prostate cancer, there are several cancer cell lines available of conducting in vitro assays. Appropriate cell lines include LNCaP, PC3, DU-145 and TSUPrl for use in cell proliferation assays.

For example, a cell line can be propagated in a standard medium, such as RPMI 1640 containing 10% fetal calf serum. Cells are first propagated and allowed to adhere. The cells can then be treated with one or more DPPP IV inhibitors at varying concentrations, and then pulsed with [³H] thymidine to evaluate incorporation, which is indicative of cell viability and proliferation. See U.S. Pat. No. 5,804,602.

It is to be understood that the description, specific examples and data, while indicating exemplary embodiments, are given by way of illustration and are not intended to limit the present invention. Various changes and modifications within the present invention will become apparent to the skilled artisan from the discussion, disclosure and data contained herein, and thus are considered part of the invention.

We claim:

1. An inhibitor of dipeptidyl peptidase IV, wherein the inhibitor comprises a proline mimetic and possesses an IC₅₀ of no more than 1 μm and has a molecular weight of no more than 500.

2. The inhibitor according to claim 1, wherein the IC₅₀ is no more than 100 nm.

3. The inhibitor according to claim 1, wherein the inhibitor can be used to treat a central nervous system disorder selected from the group consisting of strokes, tumors, ischemia, Parkinson's disease, amyotrophic lateral sclerosis and migraines.

4. A reversible inhibitor of dipeptidyl peptidase IV, wherein the inhibitor has a core structure of: 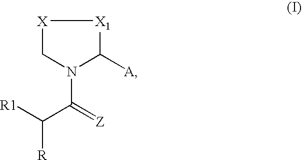
wherein:
X is CR2R3, O, S, or NR4; with the proviso that if X is S, or if X and X1 are both CH₂, and Z is O, and A is CN, and R1 is H, then R is not NH substituted with C1-C9 straight or branched chain alkyl, or NH substituted with C3-C7 cycloalkyl;
X₁ is CR2R3, O, S, or NR4 with the proviso that X and X1 cannot both be a heteroatom, and with the proviso that if X and X1 are both CH₂, and Z is O, and R1 is NH₂, then R is not 1-methylpropyl if A is COOH, and R is not cyclopentyl if A is CN;
A is H, COOH, or isosteres of carboxylic acids, such as one selected from the group consisting of CN, SO₃H, CONOH, PO₃R5R6, SO₂NHR7, tetrazole, amides, esters, and acid anhydrides, with the proviso that if A is CN, and R1 is NH₂, and Z is O, and R is 1-methylpropyl, then X and X1 are not both CH₂; X and X1 are not S; and X is not O;
Z is O or S;
R and R1 are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R and R1 can be the same or different; and
R2, R3, R4, R5, R6 and R7, if present, are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R2, R3, R4, R5, R6 and R7, if present, can be the same or different.

5. The reversible inhibitor according to claim 4, wherein the inhibitor possesses an IC₅₀ of no more than 1 μm and has a molecular weight of no more than 500.

6. A reversible inhibitor of dipeptidyl peptidase IV, wherein the inhibitor has a core structure of: 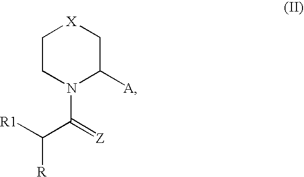
wherein:
X is CR2R3, O, S, or NR4;
A is H, COOH, or isosteres of carboxylic acids, such as one selected from the group consisting of CN, SO₃H, CONOH, PO₃R5R6, SO₂NHR7, tetrazole, amides, esters, and acid anhydrides;
Z is O or S;
R and R1 are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R and R1 can be the same or different; and
R2, R3, R4, R5, R6 and R7, if present, are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R2, R3, R4, R5, R6 and R7, if present, can be the same or different.

7. The reversible inhibitor according to claim 6, wherein the inhibitor possesses an IC₅₀ of no more than 1 μm and has a molecular weight of no more than 500.

8. A reversible inhibitor of dipeptidyl peptidase IV, wherein the inhibitor has a core structure of: 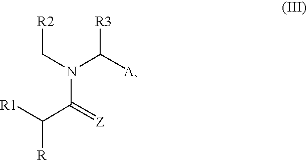
wherein:
A is H, COOH, or isosteres of carboxylic acids, such as one selected from the group consisting of CN, SO₃H, CONOH, PO₃R5R6, SO₂NHR7, tetrazole, amides, esters, and acid anhydrides;
Z is O or S;
R, R1, R2 and R3 are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R, R1, R2 and R3 can be the same or different; and
R4, R5, R6 and R7, if present, are independently selected from the group of functional groups consisting of H, C₁-C₉ branched or straight chain alkyl C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R4, R5, R6 and R7, if present, can be the same or different.

9. The reversible inhibitor according to claim 8, wherein the inhibitor possesses an IC₅₀ of no more than 1 μm and has a molecular weight of no more than 500.

10. A reversible inhibitor of dipeptidyl peptidase IV, wherein the inhibitor has a core structure of: 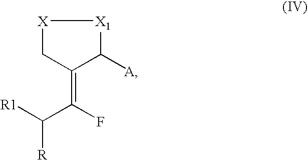
wherein:
X is CR2R3, O, S, or NR4;
X₁ is CR2R3, O, S, or NR4 with the proviso that X and X1 cannot both be a heteroatom;
A is H, COOH, or isosteres of carboxylic acids, such as one selected from the group consisting of CN, SO₃H, CONOH, PO₃R5R6, SO₂NHR7, tetrazole, amides, esters, and acid anhydrides;
R and R1 are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R and R1 can be the same or different; and
R2, R3, R4, R5, R6 and R7, if present, are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R2, R3, R4, R5, R6 and R7, if present, can be the same or different.

11. The reversible inhibitor according to claim 10, wherein the inhibitor possesses an IC₅₀ of no more than 1 μm and has a molecular weight of no more than 500.

12. A method of treating a patient having a disorder of the central nervous system, comprising administering to the patient a therapeutically effective amount of a reversible inhibitor of dipeptidyl peptidase IV, wherein the inhibitor has a core structure of: 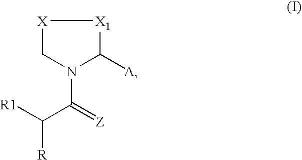
wherein:
X is CR2R3, O, S, or NR4;
X₁ is CR2R3, O, S, or NR4 with the proviso that X and X1 cannot both be a heteroatom;
A is H, COOH, or isosteres of carboxylic acids, such as one selected from the group consisting of CN, SO₃H, CONOH, PO₃R5R6, SO₂NHR7, tetrazole, amides, esters, and acid anhydrides;
Z is O or S;
R and R1 are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy,
benzyloxy, C₃-C₈ cycloalkyl, cyano, amido, thiol, trifluromethyl, or hydroxy, wherein each of R and R1 can be the same or different; and
R2, R3, R4, R5, R6 and R7, if present, are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R2, R3, R4, R5, R6 and R7, if present, can be the same or different.

13. The method according to claim 12, wherein the inhibitor possesses an IC₅₀ of no more than 1 μm and has a molecular weight of no more than 500.

14. The method according to claim 12, wherein if X is S, or if X and X1 are both CH₂, and Z is O, and A is CN, and R1 is H, then R is not NH substituted with C₁-C₉ straight or branched chain alkyl, or NH substituted with C₃-C₇ cycloalkyl; and if X and X1 are both CH₂, and Z is O, and R1 is NH₂, then R is not 1-methylpropyl if A is COOH, and R is not cyclopentyl if A is CN; and if A is CN, and R1 is NH₂, and Z is O, and R is 1-methylpropyl, then X and X1 are not both CH₂; X and X1 are not S; and X is not O;

15. A method of treating a patient having a disorder of the central nervous system, comprising administering to the patient a therapeutically effective amount of a reversible inhibitor of dipeptidyl peptidase IV, wherein the inhibitor has a core structure of: 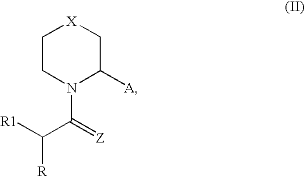
wherein:
X is CR2R3, O, S, or NR4;
A is H, COOH, or isosteres of carboxylic acids, such as one selected from the group consisting of CN, SO₃H, CONOH, PO₃R5R6, SO₂NHR7, tetrazole, amides, esters, and acid anhydrides;
Z is O or S;
R and R1 are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R and R1 can be the same or different; and
R2, R3, R4, R5, R6 and R7, if present, are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R2, R3, R4, R5, R6 and R7, if present, can be the same or different.

16. The method according to claim 15, wherein the inhibitor possesses an IC₅₀ of no more than 1 μm and has a molecular weight of no more than 500.

17. A method of treating a patient having a disorder of the central nervous system, comprising administering to the patient a therapeutically effective amount of a reversible inhibitor of dipeptidyl peptidase IV, wherein the inhibitor has a core structure of: 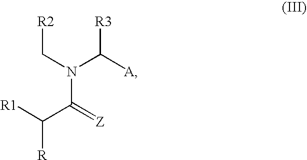
wherein
A is H, COOH, or isosteres of carboxylic acids, such as one selected from the group consisting of CN, SO₃H, CONOH, PO₃R5R6, SO₂NHR7, tetrazole, amides, esters, and acid anhydrides;
Z is O or S;
R, R1, R2 and R3 are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R, R1, R2 and R3 can be the same or different; and
R4, R5, R6 and R7, if present, are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R4, R5, R6 and R7, if present, can be the same or different.

18. The method according to claim 17, wherein the inhibitor possesses an IC₅₀ of no more than 1 μm and has a molecular weight of no more than 500.

19. A method of treating a patient having a disorder of the central nervous system, comprising administering to the patient a therapeutically effective amount of a reversible inhibitor of dipeptidyl peptidase IV, wherein the inhibitor has a core structure of: 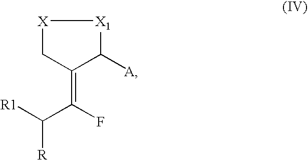
wherein:
X is CR2R3, O, S, or NR4;
X₁ is CR2R3, O, S, or NR4 with the proviso that X and X1 cannot both be a heteroatom;
A is H, COOH, or isosteres of carboxylic acids, such as one selected from the group consisting of CN, SO₃H, CONOH, PO₃R5R6, SO₂NHR7, tetrazole, amides, esters, and acid anhydrides;
R and R1 are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R and R1 can be the same or different; and
R2, R3, R4, R5, R6 and R7, if present, are independently selected from the group of functional groups consisting of H, C₁-C₉ branched or straight chain alkyl, C₂-C₉ branched or straight chain alkenyl, C₃-C₈ cycloalkyl, C₅-C₇ cycloalkenyl, aryl, heteroaryl and amino, wherein any of the functional groups can be substituted with one or more of C₁-C₉ straight or branched chain alkyl, aryl, heteroaryl, amino, halo, carbonyl, C₁-C₉ alkoxy, C₂-C₉ alkenyloxy, phenoxy, benzyloxy, C₃-C₈ cycloalkyl, cyano, amido, thiol, trifluromethyl, or hydroxy, wherein each of R2, R3, R4, R5, R6 and R7, if present, can be the same or different.

20. The method according to claim 19, wherein the inhibitor possesses an IC₅₀ of no more than 1 μm and has a molecular weight of no more than 500.

21. A method of treating a patient having a disorder of the central nervous system, comprising administering to the patient a therapeutically effective amount of a inhibitor of dipeptidyl peptidase IV.

22. The method according to claim 21, wherein the inhibitor comprises a proline mimetic and possesses an IC₅₀ of no more than 1 μm and has a molecular weight of no more than 700.

23. The method according to claim 21, wherein the inhibitor has a core structure selected from the group consisting of Core Structure I, Core Structure II, Core Structure III and Core Structure IV.

24. The method according to claim 21, wherein the inhibitor is reversible.

25. The method according to claim 21, wherein the central nervous system disorder is selected from the group consisting of strokes, tumors, ischemia, Parkinson's disease, amyotrophic lateral sclerosis and migraines.

26. A method of treating a patient having a disorder selected from the group consisting of strokes, tumors, ischemia, Parkinson's disease, memory loss, hearing loss, vision loss, migraines, brain injury, spinal cord injury, Alzheimer's disease, amyotrophic lateral, multiple sclerosis, diabetic neuropathy and prostate abnormalities, wherein the method comprises administering to the patient a therapeutically effective amount of a inhibitor of dipeptidyl peptidase IV.

27. A method according to claim 26, wherein the inhibitor comprises a proline mimetic and possesses an IC₅₀ of no more than 1 μm and has a molecular weight of no more than 700.

28. The method according to claim 26, wherein the inhibitor has a core structure selected from the group consisting of Core Structure I, Core Structure II, Core Structure III and Core Structure IV.

29. A method of using a reversible inhibitor of DPP-IV, comprising administering to a human patient suffering from a central nervous system disorder a pharamceutically effective amount of the inhibitor, wherein the inhibitor is 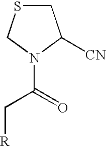
wherein R is NH—R^{I};
R^{I} is: C₁-C₁₂ straight or branched chain alkyl;
C₃-C₇ cycloalkyl;
CH₂—CH₂—NH—R^{II};
CH₂—CH₂—R^{III};
CH₂—CH₂—CHR^{IV}—R^{IV}; or
CH₂—CH₂—CH₂—R^{V};
R^{II} is a pyridine ring optionally substituted in one or two positions with halo, trifluoromethyl, cyano or nitro; or a pyrimidine ring optionally substituted in one position with halo, trifluromethyl, cyano or nitro;
R^{III} is a phenyl ring optionally substituted in one to three positions with halo or C₁-C₃ alkoxy;
Each R^{IV} is independently a phenyl ring optionally substituted in one position with halo or C₁-C₃ alkoxy; and
R^{V} is a 2-oxopyrrolidine group or a C₂-C₄ alkoxy group.

30. A method of using a reversible inhibitor of DPP-IV, comprising administering to a human patient suffering from a central nervous system disorder a pharamceutically effective amount of the inhibitor, wherein the inhibitor is 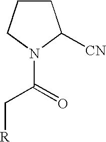
wherein R is NH—R^{I};
R^{I} is: C₁-C₁₂ straight or branched chain alkyl optionally substituted with hydroxy, acetyl,
C₁-C₃ alkoxy, or C₁-C₃ hydroxyalkyl;
C₃-C₁₂ cycloalkyl optionally substituted with hydroxyl, acetyl, C₁-C₃ alkoxy, or C₁-C₃ hydroxyalkyl;
adamantyl; indanyl; piperidyl optionally substituted with benzyl; pyrrolidine optionally substituted with benzyl; bicycloheptyl optionally substituted in one to three positions with methyl; phenyl optionally substituted with in one to three positions with halo, methoxy, trifluoromethyl; pyridyl optionally substituted in one to three positions with halo, trifluoromethyl, nitro; or pyrimidyl optionally substituted with halo, trifluoromethyl, nitro;
C₁-C₃ straight or branched chain alkyl substituted with R^{IV}, and optionally substituted with hydroxy; or
(CH₂)₁₋₃—NR^{II}R^{III};
R^{II} is hydrogen or methyl;
R^{III} is phenyl optionally substituted with CN, or pyridyl optionally substituted with CN; and
R^{IV} is a group selected from phenyl, naphthyl, cyclohexenyl, pyridyl, pyrimidyl, adamantyl, phenoxy, wherein the group is optionally substituted in one to two positions with ethoxy, methoxy, halo, phenylsulfide, or phenylsulfide substituted with hydroxymethyl.
